(19) United States
(12) Patent Application Publication
(10) Pub. No.: US 2017/0072033 A1
(43) Pub. Date: Mar. 16, 2017

(54) LIPOSOMAL COMPOSITIONS FOR MUCOSAL DELIVERY

(71) Applicant: GlaxoSmithKline Biologicals, SA, Rixensart (BE)

(72) Inventors: Nupur DUTTA, Vernon Hills, IL (US); Hardeep OBEROI, Hamilton, MT (US); David BURKHARD, Hamilton, MT (US); Jay T EVANS, Hamilton, MT (US)

(21) Appl. No.: 15/125,410

(22) PCT Filed: Mar. 12, 2015
(86) PCT No.: PCT/IB2015/051807

§ 371 Date: Sep. 12, 2016

Related U.S. Application Data
(60) Provisional application No. 61/952,124, filed on Mar. 12, 2014.

(51) Int. Cl. A61K 39/00; A61K 47/36; A61K 47/28; A61K 9/127

(52) U.S. Cl. CPC A61K 39/0011; A61K 9/1271; A61K 47/36; A61K 9/1277; A61K 47/28; A61K 2039/55555; A61K 2039/542

(57) ABSTRACT

A liposomal composition comprising lipids which form a liposomal lipid bilayer, with phospholipid-PEG conjugates incorporated into the liposomal lipid bi-layer, and a chitosan or chitosan derivative is described and claimed.

### STATEMENT REGARDING FEDERALLY SPONSORED RESEARCH

Aspects of this invention were made with United States government support pursuant to NIH Contract #HHSN272200900008C, the United States government may have certain rights in the invention

### BACKGROUND

Rapid transport of nanoparticles through human mucus has recently been reported for nanoparticles sufficiently coated with short chain polyethylene glycol (PEG, typically less than 5000 units) or certain Pluronic polymers [Cu Y, Saltzman W M. Mol Pharm. 2009; 6(1):173-181; Hanes J, et al. Nanomedicine. 2011; 6(2):365-375]. This approach, termed mucus penetration by the authors, is believed to rely on decreased mucoadhesion (rather than increasing mucoadhesion), allowing for rapid penetration of the nanoparticles through the mucus.

Toll-like receptor 4 (TLR4) modulators are immunogenic compounds used in pharmaceutical compositions and in particular as adjuvants in human vaccines. TLR-4 agonists have been formulated in liposomes for delivery via injection for vaccines. Aminoalkyl glucoseaminide phosphates (AGPs) are TLR4 modulators, some of which are particular potent and potentially reactogenic. There is a need for improved liposomal compositions in general and in particular for improved liposomal compositions of TLR4 modulators for administration of pharmaceutical compositions.

### SUMMARY OF THE INVENTION

Methods and compositions for Liposome formulations for mucosal delivery are provided.

In one embodiment the invention provides liposomal composition comprising lipids which form a liposomal lipid bilayer and further comprises phospholipid-PEG conjugates incorporated into the liposomal lipid bi-layer. Additionally the liposomal composition comprises a TLR4 agonist (e.g an AGP) and suitably comprises HEPES buffer.

In one embodiment the lipids of the liposome are DOPC in the presence of cholesterol.

In one embodiment the invention provides liposomal composition comprising lipids which form a liposomal lipid bilayer and further comprises PEG copolymers/surfactants such as poloxamers which are incorporated into the liposomal lipid bi-layer. Additionally the liposomal composition comprises TLR4 agonist (e.g. an AGP) and suitably comprises HEPES buffer. In one embodiment the lipids of the liposome are DOPC in the absence of cholesterol.

In one suitable embodiment the liposomal composition comprises chitosan or chitosan derivative.

In one suitable embodiment the invention provides a liposomal formulation comprising a DOPC liposome in the absence of sterol, poloxamers, wherein the poloxamers are incorporated into the bilayer of the DOPC liposomes, an AGP in HEPES buffer, and optionally chitosan or chitosan derivative.

In one suitable embodiment the invention provides a liposomal formulation comprising a DOPC liposome in the presence of sterol, suitably cholesterol, phospholipid-PEG conjugate wherein the phospholipid-PEG conjugate is incorporated into the bilayer of the DOPC-sterol liposome, TLR4 agonist (e.g. an AGP) in HEPES buffer, and optionally chitosan or chitosan derivative.

In one embodiment, the present invention provides a liposomal composition comprising phospholipid, phospholipid-PEG conjugate or poloxamer and an amninoalkanesulfonic buffer such as HEPES, HEPPS/EPPS, MOPS, MOBS and PIPES.

In one embodiment, the present invention provides a liposomal composition comprising phospholipid, phospholipid-PEG conjugate or poloxamer and an aminoalkyl glucosaminide phosphate (AGP), suitably CRX-601, CRX 602, CRX 527, CRX 547, CRX 526, CRX 529 or CRX 524.

In one embodiment, the present invention provides a liposomal composition comprising phospholipid, phospholipid-PEG conjugate or poloxamer AGP, amninoalkanesulfonic buffer and a chitosan or chitosan derivative, suitably chitosan oligosaccharide lactate, glycol chitosan, trimethyl chitosan or methylglycol chitosan.

In another embodiment, the present invention provides a process for improved production of a liposomal composition for sublingual delivery comprising the steps of: dissolving a lipid, such as dioleoyl phosphatidylcholine “DOPC”), phospholipid-PEG conjugate (or poloxamer in the absence of cholesterol), and AGP in organic solvent, removing the solvent to yield a phospholipid film, adding the film to HEPES buffer or HEPES buffer in saline, dispersing the film into the solution, and extruding the solution successively through polycarbonate filters to form unilamellar liposomes. The liposomal composition can additionally be aseptically filtered.

In one suitable embodiment, a liposomal composition exhibits high incorporation of a TLR4 agonist (e.g. an AGP) when the liposome is formed with cholesterol.

In another embodiment a liposomal composition exhibits high incorporation of a particular AGP, CRX 601, when the liposome is formed without a sterol such as cholesterol, providing advantages for production and formulation of such liposomal compositions, including liposomal compositions comprising poloxamer.

The liposomes of the present invention are beneficial in both the production and in the use of a pharmaceutical composition.

Additional embodiments are disclosed in the descriptions, figures and claims provided herein.

### BRIEF DESCRIPTION OF THE DRAWINGS

FIG. 1 shows stability of adjuvant-liposomes in presence methylglycol chitosan (MGC). Size/PDI and ζ-potential values with increasing concentration of MGC for adjuvant-loaded unmodified, PE-PEG2K and PE-PEG5K modified liposomes (A) & (C); and Pluronic L64, F68 and F127 modified liposomes (B) & (D). For (A) & (B), sizes are plotted as bars and PDI values as dot plot. Data are expressed as mean±SD, (n=3). Particles in the μm size range tended to precipitate over time.

FIG. 2 shows the characterization of adjuvant loaded phospholipid-PEG liposomes in presence methylglycol chitosan (MGC). Size/PDI and ζ-potential values with increasing concentration of MGC for adjuvant-loaded 1% (top row) and 25% (bottom row) PE-PEG2K and PE-PEG5K modified liposomes. Sizes are plotted as bars and PDI values as dot plot. Data are expressed as mean±SD, (n=2) for 1 mol % modification (top row) and n=1 for 25 mol % modification (bottom row). Particles in the μm size range tended to precipitate over time.

FIG. 3 shows the characterization of adjuvant loaded Pluronic liposomes in presence methylglycol chitosan (MGC). Size/PDI and ζ-potential values with increasing concentration of MGC for adjuvant-loaded 15% (top row) and 25% (bottom row) Pluronic L64, F68 and F127 modified liposomes. Sizes are plotted as bars and PDI values as dot plot. Data are expressed as mean±SD, (n=2) for 1 mol % modification (top row) and n=1 for 25 mol % modification (bottom row). Particles in the μm size range tended to precipitate over time.

FIG. 4 shows Post-secondary serum IgG titers (top), post-tertiary serum IgG titers (middle) and post-tertiary HI titers and tracheal/vaginal wash IgA titers (bottom) from phospholipid-PEG modified liposome study. Liposomes with 1, 5, and 25 mole % MPEG-2000-DSPE or MPEG-5000-DPPE substitution were evaluated at a dose of 5 μg CRX-601/animal/vaccination. The CRX-601 dose in IM control is 1 μg/animal/vaccination.

FIG. 5 shows post tertiary HI titers (A) and tracheal/vaginal wash IgA titers (B) from phospholipid-PEG modified liposome study.

FIG. 6. Post-secondary serum IgG titers (top), post-tertiary serum IgG titers (middle) and post-tertiary tracheal/vaginal wash IgA titers (bottom) from poloxamer 407 modified liposome study. Liposomes with 5, 10 and 15 mole % poloxamer 407 substitutions were evaluated at a dose of 1 or 5 μg CRX-601/animal/vaccination dose.

FIG. 7. Post-secondary serum IgG titers (top), post-tertiary serum IgG titers (middle) and post-tertiary tracheal/vaginal wash IgA titers (bottom) from poloxamers 407, 188, and 184 modified liposome study. Liposomes with 15, and 25 mole % poloxamer 407, 188, or 184 substitutions were evaluated at a dose of 5 μg CRX-601/animal/vaccination dose.

FIG. 8 shows post tertiary HI titers (A) and tracheal/vaginal wash IgA titers (B from poloxamers 407, 188, and 184 modified liposome study. In the panels the figure labels represent poloxamer 407 (F127), poloxamer 188 (F68) and poloxamer 184 (F64).

FIG. 9. Post-secondary serum IgG titers (top), post-tertiary serum IgG titers (middle) and post-tertiary tracheal/vaginal wash IgA titers (bottom) from poloxamer 407 (in figure legends indicated as F127) modified liposome+methylglycol chitosan study. Liposomes with 5, 15 or 15 mole % poloxamer 407 addition in combination with methylglycol chitosan were evaluated at a dose of 5 μg CRX-601/animal/vaccination. The CRX-601 dose in IM control is 1.5 μgs/animal/vaccination.

FIG. 10 Post-secondary serum IgG titers (A), post-tertiary serum IgG titers (B) and post-tertiary HI titers (C) and tracheal/vaginal wash IgA titers (D) from phospholipid-PEG modified liposome+methylglycol chitosan or chitosan oligosaccharide lactate study. Liposomes with 5 mole % MPEG-2000-DSPE or MPEG-5000-DPPE substitution in combination with methylglycol chitosan or chitosan oligosaccharide lactate were evaluated at a dose of 5 μg CRX-601/animal/vaccination. The CRX-601 dose in IM control is 1.5 μgs/animal/vaccination.

FIG. 11. Post-secondary serum IgG titers (top), post-tertiary serum IgG titers (middle) and post-tertiary tracheal/vaginal wash IgA titers (bottom) from poloxamer 407 (in figure legends indicated as F127) modified liposome+methylglycol chitosan study. Liposomes with 5, 15 or 15 mole % poloxamer 407 addition in combination with methylglycol chitosan were evaluated at a dose of 5 μg CRX-601/animal/vaccination. The CRX-601 dose in IM control is 1.5 μgs/animal/vaccination.

FIG. 12. Post-secondary serum IgG titers (A), post-tertiary serum IgG titers (B) and post-tertiary HI titers (C) tracheal/vaginal wash IgA titers (D) from poloxamer 407 (in figure legends indicated as F127) modified liposome+methylglycol chitosan study. Liposomes with 5, 15 or 15 mole % poloxamer 407 addition in combination with methylglycol chitosan were evaluated at a dose of 5 μg CRX-601/animal/vaccination. The CRX-601 dose in IM control is 1.5 μg/animal/vaccination.

### DETAILED DESCRIPTION OF THE INVENTION

Liposomes

The term “liposome(s)” generally refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Lipids which are capable of forming liposomes include all substances having fatty or fat-like properties. Lipids which can make up the lipids in the liposomes may be selected from the group comprising glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids.

In a particular embodiment of the invention the liposomes comprise a phospholipid. Suitable phospholipids include (but are not limited to): phosphocholine (PC) which is an intermediate in the synthesis of phosphatidylcholine; natural phospholipid derivates: egg phosphocholine, egg phosphocholine, soy phosphocholine, hydrogenated soy phosphocholine, sphingomyelin as natural phospholipids; and synthetic phospholipid derivates: phosphocholine (didecanoyl-L-α-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine DSPE 1,2-Dioleoyl-sn-Glycero-3-Phosphoethanolamine [DOPE]), phoshoserine, polyethylene glycol [PEG] phospholipid (mPEG-phospholipid, polyglycerin-phospholipid, funcitionilized-phospholipid, terminal activated-phosholipid) 1,2-dioleoyl-3-(trimethylammonium) propane (DOTAP) and Sphingomyelin (SPNG). In one embodiment the liposomes comprise 1-palmitoyl-2-oleoyl-glycero-3-phosphoethanolamine. In one embodiment highly purified phosphatidylcholine is used and can be selected from the group comprising Phosphatidylcholine (from EGG), Phosphatidylcholine Hydrogenated (from EGG), Phosphatidylcholine (from SOY) and Phosphatidylcholine Hydrogenated (from SOY). In a further embodiment the liposomes comprise phosphatidylethanolamine [POPE] or a derivative thereof.

Liposome size may vary from 30 nm to several 5 μm depending on the phospholipid composition and the method used for their preparation. In particular embodiments of the invention, the liposome size will be in the range of 30 nm to 500 nm and in further embodiments 50 nm to 200 nm, suitably less than 200 nm. Dynamic laser light scattering is a method used to measure the size of liposomes well known to those skilled in the art.

In a suitable liposomal formulation the lipid comprises dioleoyl phosphatidylcholine [DOPC] (2-Dioleoyl-sn-glycero-3-phosphocholine) and a sterol, in particular cholesterol, and optionally in the absence of sterol.

Liposomal Composition

A “liposomal composition” is a prepared composition comprising a liposome and the contents within the liposome, particularly including, but not limited to:
a) the lipids which form the liposome bilayer(s),
b) compounds other than the lipids within the bi-layer(s) of the liposome,
c) compounds within and associated with the aqueous interior(s) of the liposome, and
d) compounds bound to or associated with the outer layer of the liposome.

Thus, in addition to the lipids of the liposome, a liposomal composition of the present invention suitably may include, but is not limited to, pharmaceutically active ingredients, vaccine antigens and adjuvants, excipients, carriers mucoadhesives, mucopenetrants and buffering agents. In a preferred embodiment, such compounds are complementary to and/or are not significantly detrimental to the stability or AGP-incorporation efficiency of the liposomal composition.

“Liposomal formulation” means a liposomal composition, such as those described herein, formulated suitably with other compounds for storage and/or administration to a subject.

Thus, a “liposomal formulation” of the present invention, includes a liposomal composition as defined herein, and may additionally include, but is not limited to, liposomal compositions outside the scope of the present invention, as well as pharmaceutically active ingredients, vaccine antigens and adjuvants, excipients, carriers and buffering agents. In a preferred embodiment, such compounds are complementary to and/or are not significantly detrimental to the stability or AGP-incorporation efficiency of the liposomal composition of the present invention.

Aminoalkyl Glucosaminide Phosphate Compounds.

AGPs are Toll-Like Receptor 4 (TLR4) modulators. Toll-like receptor 4 recognizes bacterial LPS (lipopolysaccharide) and when activated initiates an innate immune response. AGPs are a monosaccharide mimetic of the lipid A protein of bacterial LPS and have been developed with ether and ester linkages on the “acyl chains” of the compound. Processes for making these compounds are known and disclosed, for example, in WO 2006/016997, U.S. Pat. Nos. 7,288,640 and 6,113,918, and WO 01/90129, which are hereby incorporated by reference in their entireties. Other AGPs and related processes are disclosed in U.S. Pat. No. 7,129,219, U.S. Pat. No. 6,525,028 and U.S. Pat. No. 6,911,434. AGPs with ether linkages on the acyl chains employed in the composition of the invention are known and disclosed in WO 2006/016997 which is hereby incorporated by reference in its entirety. Of particular interest, are the aminoalkyl glucosaminide phosphate compounds set forth and described according to Formula (III) at paragraphs [0019] through [0021] in WO 2006/016997.

Aminoalkyl glucosaminide phosphate compounds employed in the present invention have the structure set forth in Formula 1 as follows:

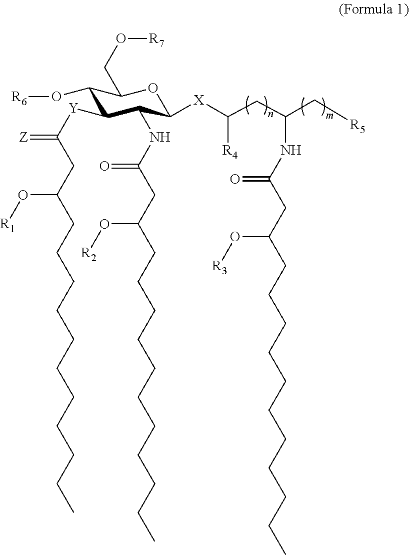

wherein
m is 0 to 6
n is 0 to 4;
X is O or S, preferably O;
Y is O or NH;
Z is O or H;
each R₁, R₂, R₃ is selected independently from the group consisting of a C₁₋₂₀ acyl and a C₁₋₂₀ alkyl;
R₄ is H or Me;
R₅ is selected independently from the group consisting of —H, —OH, —(C₁-C₄) alkoxy, —PO₃R₈R₉, —OPO₃R₈R₉, —SO₃R₈, —OSO₃R₈, —NR₈R₉, —SR₈, —CN, —NO₂, —CHO, —CO₂R₈, and —CONR₈R₉, wherein R₈ and R₉ are each independently selected from H and (C₁-C₄) alkyl; and
each R₆ and R₇ is independently H or PO₃H₂.

In Formula 1 the configuration of the 3′ stereogenic centers to which the normal fatty acyl residues (that is, the secondary acyloxy or alkoxy residues, e.g., R₁O, R₂O, and R₃O) are attached is R or S, preferably R (as designated by Cahn-Ingold-Prelog priority rules). Configuration of aglycon stereogenic centers to which R₄ and R₅ are attached can be R or S. All stereoisomers, both enantiomers and diastereomers, and mixtures thereof, are considered to fall within the scope of the present invention.

The number of carbon atoms between heteroatom X and the aglycon nitrogen atom is determined by the variable “n”, which can be an integer from 0 to 4, preferably an integer from 0 to 2.

The chain length of normal fatty acids R₁, R₂, and R₃ can be from about 6 to about 16 carbons, preferably from about 9 to about 14 carbons. The chain lengths can be the same or different. Some preferred embodiments include chain lengths where R1, R2 and R3 are 6 or 10 or 12 or 14.

Formula 1 encompasses L/D-seryl, -threonyl, -cysteinyl ether and ester lipid AGPs, both agonists and antagonists and their homologs (n=1-4), as well as various carboxylic acid bioisosteres (i.e, R₅ is an acidic group capable of salt formation; the phosphate can be either on 4- or 6-position of the glucosamine unit, but preferably is in the 4-position).

In a preferred embodiment of the invention employing an AGP compound of Formula 1, n is 0, R₅ is CO₂H, R₆ is PO₃H₂, and R₇ is H. This preferred AGP compound is set forth as the structure in Formula 1a as follows:

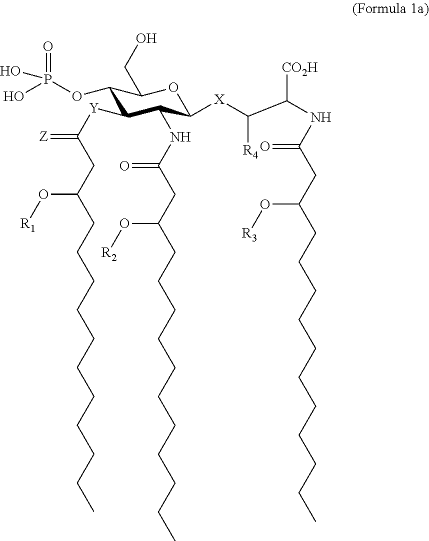

wherein X is O or S; Y is O or NH; Z is O or H; each R₁, R₂, R₃ is selected independently from the group consisting of a C₁₋₂₀ acyl and a C₁₋₂₀ alkyl; and R₄ is H or methyl.

In Formula 1a the configuration of the 3′ stereogenic centers to which the normal fatty acyl residues (that is, the secondary acyloxy or alkoxy residues, e.g., R₁O, R₂O, and R₃O) are attached as R or S, preferably R (as designated by Cahn-Ingold-Prelog priority rules). Configuration of aglycon stereogenic centers to which R₄ and CO₂H are attached can be R or S. All stereoisomers, both enantiomers and diastereomers, and mixtures thereof, are considered to fall within the scope of the present invention.

Formula 1a encompasses L/D-seryl, -threonyl, -cysteinyl ether or ester lipid AGPs, both agonists and antagonists.

In both Formula 1 and Formula 1a, Z is O attached by a double bond or two hydrogen atoms which are each attached by a single bond. That is, the compound is ester-linked when Z═Y═O; amide-linked when Z═O and Y═NH; and ether-linked when Z═H/H and Y═O.

Especially preferred compounds of Formula 1 are referred to as CRX-601 and CRX-527. Their structures are set forth as follows:

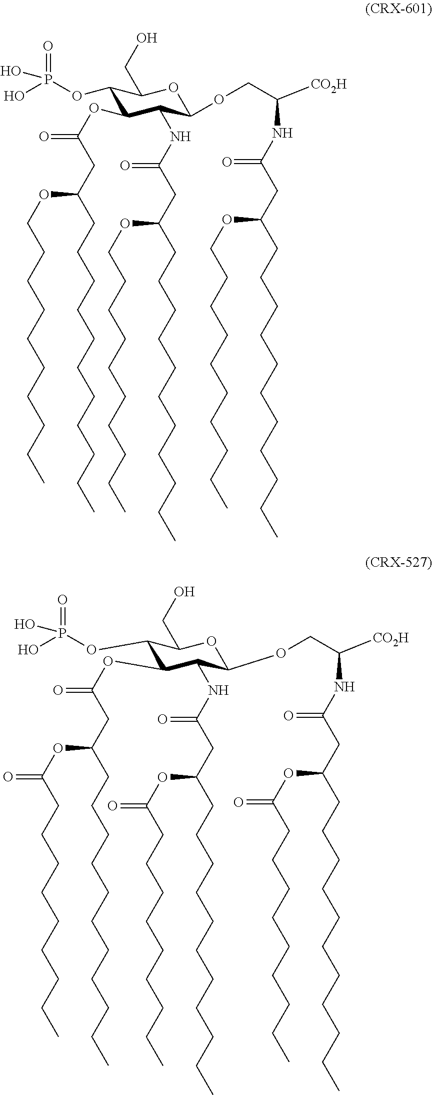

Additionally, another preferred embodiment employs CRX 547 having the structure shown.

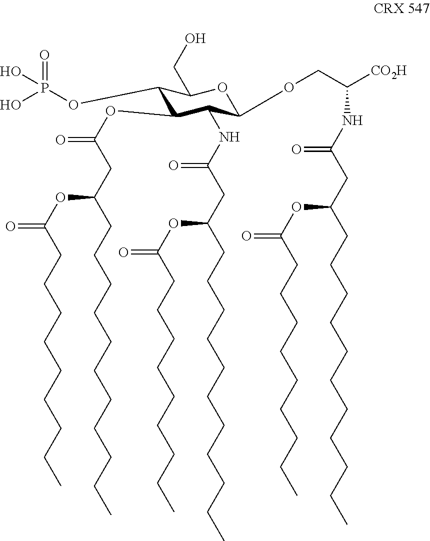

Still other embodiments include AGPs such as CRX 602 or CRX 526 providing increased stability to AGPs having shorter secondary acyl or alkyl chains.

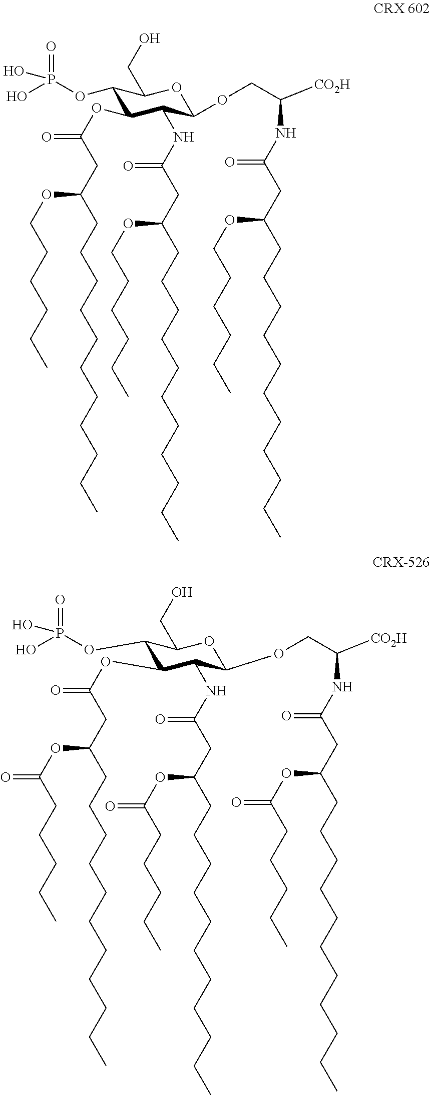

Other AGPs suitable for use in the present invention include CRX 524 and CRX 529.

Buffers.

In one embodiment of the present invention, a liposomal composition is buffered using a zwitterionic buffer. Suitably, the zwitterionic buffer is an aminoalkanesulfonic acid or suitable salt. Examples of amninoalkanesulfonic buffers include but are not limited to HEPES, HEPPS/EPPS, MOPS, MOBS and PIPES. Preferably, the buffer is a pharmaceutically acceptable buffer, suitable for use in humans, such as in for use in a commercial injection product. Most preferably the buffer is HEPES. The liposomal composition may suitable include an AGP.

In suitable embodiments of the present invention the liposomes are buffered using a buffer selected from the group consisting of:
i) HEPES having a pH of about 7,
ii) citrate (e.g., sodium citrate) having a pH of about 5, and
iii) acetate (e.g., ammonium acetate) having a pH of about 5.

In a preferred embodiment of the present invention the AGPs CRX-601, CRX-527 and CRX-547 are included in a liposomal composition buffered using HEPES having a pH of about 7. The buffers may be used with an appropriate amount of saline or other excipient to achieve desired isotonicity. In one preferred embodiment 0.9% saline is used.

HEPES: CAS Registry Number: 7365-45-9 C₈H₁₈N₂O₄S

1-Piperazineethanesulfonic acid, 4-(2-hydroxyethyl)-

HEPES is a zwitterionic buffer designed to buffer in the physiological pH range of about 6 to about 8 (e.g. 6.15-8.35) and more specifically from a more useful range of about 6.8 to about 8.2 and, as in the present invention, between about 7 and about 8 or between 7 and 8, and preferably between about 7 and less than 8. HEPES is typically a white crystalline powder and has the molecular formula: C₈H₁₈N₂O₄S of the following structure:

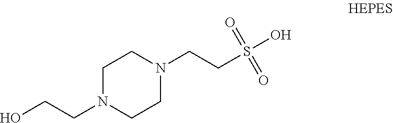 [SMILES: O=S(=O)(O)CCN1CCN(CCO)CC1]

HEPES is well-known and commercially available. (See, for example, Good et al., Biochemistry 1966.)

PEG

Polyethylene glycol (PEG), also known as polyethylene oxide (PEO) or polyoxyethylene (POE) is a hydrophilic polymer (polyether) with many applications ranging from industrial manufacturing to medicine. This polymer is inexpensive, has good biocompatibility and has been approved for internal applications in humans by regulatory agencies. PEG chains of molecular weights ranging from 1 to 15 kDa have been widely employed as steric protectors in various colloidal systems. Owing to its high aqueous solubility, high mobility and large exclusion volume, hydrated PEG forms a dense brush of polymer chains stretching out and covering the particle surface. This minimizes the interfacial free energy of the particle surface and impedes its interaction with other particles, providing colloidal stability to the system. The ability of PEG coating to prevent interaction with proteins and other biomolecules in blood, and cells has widely been utilized to prolong the circulation time of drug carriers in the blood, reduce particle opsonization and to make them less recognizable by the reticuloendothelial system (RES) in the liver and the spleen. In particular for delivery via the mucosal route, PEG, depending on its chain length has been shown to possess both muco-adhesive (long chain) and muco-inert (short chain) properties

Surface modification of colloidal drug carriers, in particular liposomes, with PEG can be achieved in several ways: 1) using amphiphilic PEG-lipid conjugates, PEG copolymers such as poloxamers, or other such PEG-hydrophobe conjugates, either physically adsorbing them onto the surface of the vesicles, or by incorporating them during liposome preparation, or 2) by covalently grafting PEG chains with a terminal functional groups to reactive groups on the surface of preformed liposomes.

Phospholipid-PEG Conjugates

Conjugates of PEG with phospholipids have widely been employed for incorporating PEG onto liposomes. The phospholipid part acts as an anchor by embedding into the hydrophobic interior of the bilayer and grafts the PEG chain to the liposome aqueous surface. These conjugates have excellent biocompatibility. Several different conjugates, depending upon the PEG chain length and the type of phospholipid used are available. Doxil, a clinically approved liposomal doxorubicin formulation, and many other liposomal formulations in late stage clinical trials (such as Lipoplatin, SPI-77, Lipoxal, etc.) are based on this concept of incorporating PEG-phospholipids.

Numerous phospholipid-PEG conjugates are known in the art and many phospholipid-PEG conjugates are commercially available, such as:

MPEG-2000-DSPE: N-(Carbonyl-methoxypolyethylenglycol-2000)-1,2-distearoyl-sn-glycero-3-phosphoethanolamine sodium salt,

MPEG-5000-DPPE: N-(Carbonyl-methoxypolyethylenglycol-5000)-1,2-dipalmitoyl-sn-glycero-3-phosphoethanolamine sodium salt.

Other related phospholipid-PEG conjugates include, but are not limited to:

DPPE-mPEG(1000): 1,2-dipalmitoyl-sn-glycero-3-phosphoethanolamine-N-[methoxy(polyethylene glycol)-1000] (ammonium salt);

DSPE-mPEG(1000): 1,2-distearoyl-sn-glycero-3-phosphoethanolamine-N-[methoxy(polyethylene glycol)-1000] (ammonium salt);

DOPE-mPEG(1000): 1,2-dioleoyl-sn-glycero-3-phosphoethanolamine-N-[methoxy(polyethylene glycol)-1000] (ammonium salt);

DPPE-mPEG(2000): 1,2-dipalmitoyl-sn-glycero-3-phosphoethanolamine-N-[methoxy(polyethylene glycol)-2000] (ammonium salt)DOPE-mPEG(2000): 1,2-dioleoyl-sn-glycero-3-phosphoethanolamine-N-[methoxy(polyethylene glycol)-2000] (ammonium salt);

DSPE-mPEG(5000): 1,2-distearoyl-sn-glycero-3-phosphoethanolamine-N-[methoxy(polyethylene glycol)-5000] (ammonium salt); and

DOPE-mPEG(5000): 1,2-dioleoyl-sn-glycero-3-phosphoethanolamine-N-[methoxy(polyethylene glycol)-5000] (ammonium salt).

Poloxamers

Poloxamers are amphiphilic, nonionic triblock copolymers composed of a central hydrophobic chain of polyoxypropylene (poly(propylene oxide)) flanked by two hydrophilic PEG chains. Poloxamers are also known by the trade names Synperonics, Pluronics and Kolliphor. The lengths of the polymer blocks can be customized, thus many different poloxamers exist, differing in their properties and can exist as liquids, pastes or solids. Because of their amphiphilic structure, these polymers have surfactant properties which can be used to emulsify water-insoluble substances, form supramolecular associates (micelles or vesicles) in water solutions that can trap various compounds, or can be incorporated into other colloidal particles such as liposomes. A characteristic feature of these synthetic polymers is a relatively low toxicity and biological compatibility. For this reason, these polymers are commonly used in industrial applications, cosmetics, and pharmaceuticals. They have also been utilized in therapy for burns and wounds, cryoprotectants, drug emulsifiers, vaccine adjuvants, in medical imaging, management of vascular diseases and have been shown to sensitize drug resistant cancers to chemotherapy. The central hydrophobic block is essential for the incorporation of poloxamers into liposome bilayers and other colloidal drug delivery particles.

Pharmaceutically acceptable poloxamers include, but are not limited to:

poloxamer 407 (Pluronic® F127);

poloxamer 184 (Pluronic® L64) and

poloxamer 188 (Pluronic® L68)

Pluronic is a registered trademark of BASF.

Numerous other poloxamers are commercially available.

Chitosan

Chitosan is a natural cationic polysaccharide derived from chitin by partially deacetylating its acetamido groups under strong alkaline solutions. Over the last two decades, chitosan has found widespread use in biomedical and drug delivery applications due to its low toxicity, good biocompatibility and excellent mucoadhesive properties (van der Lubben, I. M., Verhoef, J. C., Borchard, G. & Junginger, H. E. Chitosan for mucosal vaccination. Adv. Drug Deliv. Rev. 52, 139-144, 2001). Mucosal adhesion of chitosan is believed to involve complex mechanisms, with electrostatic interaction between cationic chitosan and the anionic mucin coating on the mucosal surface being the primary factor, although hydrogen bonding and hydrophobic effects are also believed to play a significant role.

Underivatized (“nonderivatized”) chitosan has limited solubility (˜1 mg/mL) and is soluble only under acidic conditions (pH<6.5). Derivatives of chitosan such as glycol chitosan, methylglycol chitosan, and chitosan oligosaccharide lactate however have significantly improved solubility (˜10 mg/mL) at physiological pH.

Commercially available chitosan derivatives include, but are not limited to; chitosan oligosaccharide lactate, glycol chitosan, or methylglycol chitosan (MGC). These derivatives have varying physical properties from chitosan which may make them more suitable for use with antigens, adjuvants, liposomes and the like.

Liposome Preparation

Standard methods for making liposomes include, but are not limited to methods reported in Liposomes: A Practical Approach, V. P. Torchilin, Volkmar Weissig Oxford University Press, 2003 and are well known in the art.

In one suitable process for making a liposomal composition of the present invention, an AGP (e.g. CRX-601 (0.2% w/v)) and DOPC (specifically, 1,2-Dioleoyl-sn-glycero-3-phosphocholine, 3-4% w/v)) and optionally a sterol (e.g. cholesterol (1% w/v)) are dissolved as in an organic phase of chloroform or tetrahydrofuran in a round bottom flask. The organic solvent is removed by evaporation on a rotary evaporator and further with high pressure vacuum for 12 hrs. To the mixed phospholipid film thus obtained is added 10 ml of an aminoalkanesulfonic buffer such as 10 mM HEPES or 10 mM HEPES-Saline buffer pH 7.2. The mixture is sonicated on a water bath (20-30° C.) with intermittent vortexing until all the film along the flask walls is dispersed into the solution (30 min-1.5 hrs). The solution is then extruded successively through polycarbonate membrane filters with the aid of a Lipid extruder (Northern Lipids Inc., Canada) to form unilamellar liposomes. The liposome composition is then aseptically filtered using a 0.22 μm filter into a sterile depyrogenated container. The average particle size of the resultant formulation as measured by dynamic light scattering is 80-120 nm with a net negative zeta-potential. The formulation represents final target concentrations of 2 mg/mL CRX-601, 10 mg/mL cholesterol, and 40 mg/mL total phospholipids.

Suitably a PEG-phospholipid (e.g. MPEG-2000-DSPE (0.1-3% w/v) or MPEG-5000-DPPE (0.3-6% w/v)) or a poloxomer (e.g. poloxamer 407 (1-16% w/v)) is dissolved with the AGP and DOPC lipids at the outset of the process. Suitably, the liposomal composition is formulations can be mixed with an aseptic solution of chitosan (e.g. MGC 200 mg) dissolved in HEPES.

The aminoalkyl glucosaminide 4-phosphate (AGP) CRX-601 used in this work can be synthesized as described previously {Bazin, 2008 32447/id}, and purified by chromatography (to >95% purity). CRX-601, either in the starting material or in the final product can be quantified by a standard reverse phase HPLC analytical method.

In one embodiment during the preparation of liposomes, CRX-601 formulated in the HEPES buffer (pH=7.0) obtains the desired reduction of particle size five times faster, as compared to liposome hydration buffer (“LHB,” phosphate based, pH=6.1). The rehydration of the CRX-601 lipid films in the HEPES buffer requires four times less total pressure and time to formulate the liposomes as compared to the LHB phosphate buffer. This is a significant improvement since it saves both energy and time and puts much less stress on the AGPs during the processing of the liposomes.

Suitable ranges (w/v) of components of liposome composition include one embodiment comprising a Lipid in a range of about 3-4% w/v, a sterol at 1% w/v, an active, such as an AGP in range of 0.1-1% w/v and an aminoalkanesulfonic buffer at 10 mM. In one embodiment sterol is suitably present a range of 0.5-4% w/v. Additionally in one embodiment the lipid:sterol:active ratio is about 3-4:1:0.1-1.

### EXAMPLES

### Preparation of Modified DOPC Liposomes with CRX-601

### Example 1

### Liposomes with 1 mol % MPEG-2000-DSPE Substitution

The mol % substitution in this example refers to the amount of MPEG-2000-DSPE relative to the total phospholipid content. The CRX-601 (20 mg), 1,2-Dioleoyl-sn-glycero-3-phosphocholine, abbreviated as DOPC (396 mg), cholesterol (100 mg) and the PEG phospholipid [N-(Carbonyl-methoxypolyethylenglycol-2000)-1,2-distearoyl-sn-glycero-3-phosphoethanolamine sodium salt, abbreviated as MPEG-2000-DSPE (15 mg) were dissolved in an organic phase of tetrahydrofuran in a round bottom flask. The organic solvent was removed by evaporation on a rotary evaporator and further with high pressure vacuum for 12 hrs. To the mixed phospholipid film thus obtained was added 10 ml of 10 mM HEPES or 10 mM HEPES-Saline buffer pH 7.2. The mixture was sonicated on a water bath (20-30° C.) with intermittent vortexing until all the film along the flask walls was dispersed into the solution (30 min-1.5 hrs). The solution was then extruded successively through polycarbonate filters having a pore size of 600 nm (1 pass), 400 nm (1 pass), and 200 nm (2-4 passes) with the aid of a lipid miniextruder (Lipex™ extruder (Northern Lipids Inc., Canada)) to form unilamellar liposomes. The liposome composition was then aseptically filtered using a 0.22 μm filter into a sterile depyrogenated container. The average particle size of the resultant formulation as measured by dynamic light scattering was 80-120 nm with a net negative zeta-potential. The formulation represents final target concentrations of 2 mg/mL CRX-601, 10 mg/mL cholesterol, and 40 mg/mL total phospholipids.

The aminoalkyl glucosaminide 4-phosphate (AGP) CRX-601 used in this work was synthesized as described previously {Bazin, 2008 32447/id}, and purified by chromatography (to >95% purity). CRX-601, either in the starting material or in the final product was quantified by a standard reverse phase HPLC analytical method.

### Example 2

### Liposomes with Higher mol % MPEG-2000-DSPE Substitution

The liposome formulations were prepared as in Example 1 but with varying DOPC and MPEG-2000-DSPE amounts to obtain the desired mol % substitution. Formulations with targeted substitutions of 5, 10, 15, and 25 mol % had been prepared. Representative average particle sizes and zeta-potential as measured by dynamic light scattering are shown in Table 1. Formulations with higher than 25 mol % substitutions are difficult to prepare, limited by dissolution of the lipid film into the buffer during sonication, as solubility limit of the components approaches. At high substitutions, PEG phospholipids are expected to be saturating the liposome bilayer with excess being in solution as micelles or unimers.

TABLE 1
Representative formulation parameters for MPEG-
2000-DSPE modified DOPC-Cholesterol liposomes.

| | Z-Ave size | | ZP |
|---|---|---|---|
| Formulation | (nm) | PDI | (mV) |
| PE-PEG2000 aqueous solution | 6 | 0.34 | −15 |
| 1 mole % MPEG-2000-DSPE CRX-601 | 83 | 0.19 | −12 |
| DOPC-Chol liposome | | | |
| 5 mole % MPEG-2000-DSPE CRX-601 | 120 | 0.20 | −8 |
| DOPC-Chol liposome | | | |
| 15 mole % MPEG-2000-DSPE CRX-601 | 110 | 0.22 | −22 |
| DOPC-Chol liposome | | | |
| 25 mole % MPEG-2000-DSPE CRX-601 | 110 | 0.22 | −7 |
| DOPC-Chol liposome | | | |
| 30 mole % MPEG-2000-DSPE CRX-601 | 90 | 0.20 | −15 |
| DOPC-Chol liposome | | | |

### Example 3

### Liposomes with 1 mol % MPEG-5000-DPPE Substitution

The liposome formulation was prepared as in Example 1 but with PEG phospholipid N-(Carbonyl-methoxypolyethylenglycol-5000)-1,2-dipalmitoyl-sn-glycero-3-phosphoethanolamine sodium salt, abbreviated as MPEG-5000-DPPE (33 mg) used instead of MPEG-2000-DSPE. The average particle size of the resultant formulation as measured by dynamic light scattering was 80-120 nm.

### Example 4

### Liposomes with Higher mol % MPEG-5000-DPPE Substitution

The liposome formulations were prepared as in Example 3 but with varying DOPC and MPEG-5000-DPPE amounts to obtain the desired mol % substitution. Formulations with targeted substitutions of 5, 10, 15, and 25 mol % had been prepared. Representative average particle sizes and zeta-potential as measured by dynamic light scattering are shown in Table 2. Formulations with higher than 25 mol % substitutions are difficult to prepare, limited by dissolution of the lipid film into the buffer during sonication, as solubility limit of the components approaches. At high substitutions, PEG phospholipids are expected to be saturating the liposome bilayer with excess being in solution as micelles or unimers.

TABLE 2
Representative formulation parameters for MPEG-
5000-DPPE modified DOPC-Cholesterol liposomes.

| | Z-Ave size | | ZP |
|---|---|---|---|
| Formulation | (nm) | PDI | (mV) |
| PE-PEG5000 aqueous solution | variable | 1.0 | |
| 1 mole % PE-PEG5000 CRX-601 | 90 | 0.20 | −3 |
| DOPC-Chol liposome | | | |
| 5 mole % PE-PEG5000 CRX-601 | 100 | 0.20 | −2 |
| DOPC-Chol liposome | | | |
| 15 mole % PE-PEG5000 CRX-601 | 100 | 0.19 | −7 |
| DOPC-Chol liposome | | | |
| 25 mole % PE-PEG5000 CRX-601 | 75 | 0.22 | −2 |
| DOPC-Chol liposome | | | |
| 30 mole % PE-PEG5000 CRX-601 | 80 | 0.23 | −7 |
| DOPC-Chol liposome | | | |

### Example 5

### Preparation of Liposomes with 1 mol % Poloxamer 407 Addition

The mol % addition in this example refers to the amount of poloxamer relative to the total phospholipid content. The CRX-601 (20 mg), DOPC (400 mg), and poloxamer 407 (64 mg) were dissolved in a organic phase of tetrahydrofuran in a round bottom flask and processed as discussed in Example 1. No cholesterol was included in these preparations as it has been reported to reduce incorporation of poloxamers into the phospholipid bilayer. The average particle size of the resultant formulation as measured by dynamic light scattering was 120-180 nm. The formulation represents final target concentrations of 2 mg/mL CRX-601, 40 mg/mL DOPC, and 1 mol % (wrt DOPC) poloxamer 407.

### Example 6

### Preparation of Liposomes with Higher mol % Poloxamer 407 Addition

The liposome formulations were prepared as in Example 5 but with increasing amounts of poloxamer 407. Formulations with targeted substitutions of 5, 10, 15, and 25 mol % had been prepared. Representative average particle sizes and zeta-potential as measured by dynamic light scattering are shown in Table 3. Formulations with higher than 25 mol % additions are difficult to prepare, limited by dissolution of the lipid-poloxamer film into the buffer during sonication, as solubility limit of the components approaches. At high substitutions poloxamer 407 is expected to be saturating the liposome bilayer with excess being in solution as micelles or unimers.

TABLE 3
Representative formulation parameters for
poloxamer 407 modified DOPC liposomes.

| | Z-Ave size | | ZP |
|---|---|---|---|
| Formulation | (nm) | PDI | (mV) |
| F127 aqueous solution, | 10 | 0.35 | NT |
| 10 mg/mL | | | |
| 1 mole % F127/CRX-601 | 120 | 0.24 | −41 |
| DOPC liposome | | | |
| 1 mole % F127/CRX-601 | 110 | 0.24 | −41 |
| DOPC-Chol liposome | | | |
| 5 mole % F127 DOPC | 100 | 0.25 | −11 |
| liposome | | | |
| 5 mole % F127/CRX-601 | 100 | 0.30 | −28 |
| DOPC liposome | | | |
| 5 mole % F127/CRX-601 | 95 | 0.30 | −31 |
| DOPC-Chol liposome | | | |
| 10 mole % F127/CRX-601 | 125 | 0.2 | −40 |
| DOPC liposome | | | |
| 15 mole % F127 DOPC | 125 | 0.20 | −9 |
| liposome | | | |
| 15 mole % F127/CRX-601 | 90 | 0.24 | −37 |
| DOPC liposome | | | |

| | |
|---|---|
| 30 mole % F127/CRX-601 | Thin film did not disperse in hydration |
| DOPC liposome | buffer, solubility limit of F127 |

### Example 7

### Preparation of Liposomes with Poloxamer 188 Addition

The liposome formulations were prepared as in Example 6 but with poloxamer 188 instead of poloxamer 407. Formulations with targeted substitutions of 15 and 25 mol % have been prepared. Representative average particle sizes and zeta-potential as measured by dynamic light scattering are shown in Table 4.

### Example 8

### Preparation of Liposomes with Poloxamer 184 Addition

The liposome formulations were prepared as in Example 6 but with poloxamer 184 instead of poloxamer 407. Formulations with targeted substitutions of 15 and 25 mol % have been prepared. Representative average particle sizes and zeta-potential as measured by dynamic light scattering are shown in Table 4.

TABLE 4
Representative formulation parameters for poloxamer
188 or poloxamer 184 modified DOPC liposomes.

| | Z-ave size | ZP |
|---|---|---|
| Formulation | (nm)/PDI | (mV) |
| Blank 25 mole % Pluronic F127 DOPC liposomes | 155/0.18 | −0.6 |
| Blank 25 mole % Pluronic F68 DOPC liposomes | 176/0.12 | −29 |
| Blank 25 mole % Pluronic L64 DOPC liposomes | 75/0.21 | −0.4 |
| CRX-601 15 mole % Pluronic F127 DOPC liposomes | 161/0.21 | −0.1 |
| CRX-601 25 mole % Pluronic F127 DOPC liposomes | 122/0.19 | −3 |
| CRX-601 15 mole % Pluronic F68 DOPC liposomes | 184/0.11 | −27 |
| CRX-601 25 mole % Pluronic F68 DOPC liposomes | 180/0.10 | −22 |
| CRX-601 15 mole % Pluronic L64 DOPC liposomes | 68/0.25 | −11 |
| CRX-601 25 mole % Pluronic L64 DOPC liposomes | 88/0.22 | −5 |

### Example 9

### Preparation of Liposome Formulation with Chitosan

Methylglycol chitosan (chitosan glycol trimethyl ammonium iodide, 200 mg) was dissolved in 10 ml of 10 mM HEPES-Saline buffer pH 7.2 to yield a concentration of 20 mg/ml. The solution was aseptically filtered using a 0.22 μm filter into a sterile depyrogenated container. The formulations from example 1-4 were mixed aseptically with varying volumes of methylglycol chitosan solution to yield concentrations ranging from 1-10 mg/mL. While conventional DOPC-cholesterol liposome formulations aggregate upon mixing with chitosan or its derivatives including methylglycol chitosan (Table 5), modified liposomes (Example 1-4) reported here remain stable in suspension (Table 6). Representative average particle sizes and zeta-potential as measured by dynamic light scattering shown in Table 6, indicate some increase in particle size and a reversal in zeta-potential (net positive potential from a net negative potential) exceeding approximately 1 mg/mL methylglycol chitosan, consistent with surface coating with methylglycol chitosan. At concentrations exceeding a certain threshold, methylglycol chitosan is expected to be saturating the liposome bilayer, with excess being in solution.

TABLE 5
Representative formulation parameters indicating aggregation
of unmodified liposomal DOPC formulation with varying
concentrations of methylglycol chitosan (MGC). CRX-601
concentration is 1 mg/mL in all cases.

| | Z-Ave Size | ZP |
|---|---|---|
| Formulation | (nm)/PDI | (mV) |
| 601 DOPC-Chol liposomes | 142/0.16 | −14 |
| 601 DOPC-Chol liposomes + MGC, 0.2 mg/mL | 26350/1.0 | −8 |
| 601 DOPC-Chol liposomes + MGC, 0.4 mg/mL | 18200/1.0 | −1 |
| 601 DOPC-Chol liposomes + MGC, 0.6 mg/mL | 29800/0.73 | +3 |
| 601 DOPC-Chol liposomes + MGC, 0.8 mg/mL | 26670/0.29 | −1 |
| 601 DOPC-Chol liposomes + MGC, 1 mg/mL | 392/0.64 | +9 |
| 601 DOPC-Chol liposomes + MGC, 2 mg/mL | 212/0.61 | +7 |
| 601 DOPC-Chol liposomes + MGC, 3 mg/mL | 210/0.67 | +8 |
| 601 DOPC-Chol liposomes + MGC, 4 mg/mL | 247/0.73 | +12 |
| 601 DOPC-Chol liposomes + MGC, 5 mg/mL | 254/0.55 | +11 |
| 601 DOPC-Chol liposomes + MGC, 10 mg/mL | 231/0.32 | +21 |

TABLE 6
Representative formulations parameters for methylglycol chitosan (MGC) coated MPEG-2000-DSPE
and MPEG-5000-DPPE modified DOPC-Cholesterol liposomes at 1, 5, 15 and 30% liposome modification
prepared in 10 mM HEPES-Saline. CRX-601 concentration is 1 mg/mL in all cases.

| | Z-Ave Size | ZP |
|---|---|---|
| Formulations | (nm)/PDI | (mV) |
| CRX-601/1 mole % MPEG-2000-DSPE DOPC-Chol liposomes | 94/0.23 | −17.6 |
| CRX-601/1 mole % MPEG-2000-DSPE DOPC-Chol liposomes + MGC, 0.2 mg/mL | 183/0.39 | −2.7 |
| CRX-601/1 mole % MPEG-2000-DSPE DOPC-Chol liposomes + MGC, 0.4 mg/mL | 13990/0.9 | −2.3 |
| CRX-601/1 mole % MPEG-2000-DSPE DOPC-Chol liposomes + MGC, 0.6 mg/mL | 60670/1.0 | +2.6 |
| CRX-601/1 mole % MPEG-2000-DSPE DOPC-Chol liposomes + MGC, 0.8 mg/mL | 248/0.35 | +14.4 |
| CRX-601/1 mole % MPEG-2000-DSPE DOPC-Chol liposomes + MGC, 1 mg/mL | 103/0.34 | +4.4 |
| CRX-601/1 mole % MPEG-2000-DSPE DOPC-Chol liposomes + MGC, 2 mg/mL | 107/0.24 | +4.5 |
| CRX-601/1 mole % MPEG-2000-DSPE DOPC-Chol liposomes + MGC, 3 mg/mL | 107/0.25 | +2.3 |
| CRX-601/1 mole % MPEG-2000-DSPE DOPC-Chol liposomes + MGC, 4 mg/mL | 117/0.34 | +5.9 |
| CRX-601/1 mole % MPEG-2000-DSPE DOPC-Chol liposomes + MGC, 5 mg/mL | 107/0.26 | +7.4 |
| CRX-601/1 mole % MPEG-2000-DSPE DOPC-Chol liposomes + MGC, 10 mg/mL | 127/0.26 | +4.2 |
| CRX-601/1 mole % MPEG-5000-DPPE DOPC-Chol liposomes | 100/0.23 | −3.7 |
| CRX-601/1 mole % MPEG-5000-DPPE DOPC-Chol liposomes + MGC, 0.2 mg/mL | 123/0.17 | −1.9 |
| CRX-601/1 mole % MPEG-5000-DPPE DOPC-Chol liposomes + MGC, 0.4 mg/mL | 154/0.23 | −2.2 |
| CRX-601/1 mole % MPEG-5000-DPPE DOPC-Chol liposomes + MGC, 0.6 mg/mL | 141/0.41 | −0.9 |
| CRX-601/1 mole % MPEG-5000-DPPE DOPC-Chol liposomes + MGC, 0.8 mg/mL | 115/0.26 | +4.3 |
| CRX-601/1 mole % MPEG-5000-DPPE DOPC-Chol liposomes + MGC, 1 mg/mL | 109/0.26 | +5.8 |
| CRX-601/1 mole % MPEG-5000-DPPE DOPC-Chol liposomes + MGC, 2 mg/mL | 111/0.22 | +3.9 |
| CRX-601/1 mole % MPEG-5000-DPPE DOPC-Chol liposomes + MGC, 3 mg/mL | 110/0.23 | +0.5 |
| CRX-601/1 mole % MPEG-5000-DPPE DOPC-Chol liposomes + MGC, 4 mg/mL | 122/0.27 | +2.1 |
| CRX-601/1 mole % MPEG-5000-DPPE DOPC-Chol liposomes + MGC, 5 mg/mL | 117/0.22 | +4.1 |
| CRX-601/1 mole % MPEG-5000-DPPE DOPC-Chol liposomes + MGC, 10 mg/mL | 127/0.25 | +3.1 |
| CRX-601/5 mol % MPEG-2000-DSPE DOPC-Chol liposomes | 121/0.25 | −7.9 |
| CRX-601/5 mol % MPEG-2000-DSPE DOPC-Chol liposomes + MGC, 0.2 mg/mL | 137/0.26 | −8.0 |
| CRX-601/5 mol % MPEG-2000-DSPE DOPC-Chol liposomes + MGC, 0.4 mg/mL | 136/0.23 | −3.8 |
| CRX-601/5 mol % MPEG-2000-DSPE DOPC-Chol liposomes + MGC, 0.6 mg/mL | 142/0.28 | −5.0 |
| CRX-601/5 mol % MPEG-2000-DSPE DOPC-Chol liposomes + MGC, 0.8 mg/mL | 131/0.27 | −1.2 |
| CRX-601/5 mol % MPEG-2000-DSPE DOPC-Chol liposomes + MGC, 1 mg/mL | 140/0.27 | +0.3 |
| CRX-601/5 mol % MPEG-2000-DSPE DOPC-Chol liposomes + MGC, 2 mg/mL | 135/0.27 | +3.7 |
| CRX-601/5 mol % MPEG-2000-DSPE DOPC-Chol liposomes + MGC, 3 mg/mL | 143/0.28 | +7.0 |
| CRX-601/5 mol % MPEG-2000-DSPE DOPC-Chol liposomes + MGC, 4 mg/mL | 146/0.29 | +13.4 |
| CRX-601/5 mol % MPEG-2000-DSPE DOPC-Chol liposomes + MGC, 5 mg/mL | 164/0.27 | +3.6 |
| CRX-601/5 mol % MPEG-2000-DSPE DOPC-Chol liposomes + MGC, 10 mg/mL | 165/0.28 | +11.7 |
| CRX-601/5 mol % MPEG-5000-DPPE DOPC-Chol liposomes | 97/0.17 | −4.9 |
| CRX-601/5 mol % MPEG-5000-DPPE DOPC-Chol liposomes + MGC, 0.2 mg/mL | 102/0.16 | −3.1 |
| CRX-601/5 mol % MPEG-5000-DPPE DOPC-Chol liposomes + MGC, 0.4 mg/mL | 103/0.20 | −3.9 |
| CRX-601/5 mol % MPEG-5000-DPPE DOPC-Chol liposomes + MGC, 0.6 mg/mL | 106/0.17 | −2.0 |
| CRX-601/5 mol % MPEG-5000-DPPE DOPC-Chol liposomes + MGC, 0.8 mg/mL | 107/0.21 | −1.3 |
| CRX-601/5 mol % MPEG-5000-DPPE DOPC-Chol liposomes + MGC, 1 mg/mL | 109/0.19 | 0.1 |
| CRX-601/5 mol % MPEG-5000-DPPE DOPC-Chol liposomes + MGC, 2 mg/mL | 103/0.19 | −0.9 |
| CRX-601/5 mol % MPEG-5000-DPPE DOPC-Chol liposomes + MGC, 3 mg/mL | 107/0.19 | −0.9 |
| CRX-601/5 mol % MPEG-5000-DPPE DOPC-Chol liposomes + MGC, 4 mg/mL | 108/0.23 | +3.1 |
| CRX-601/5 mol % MPEG-5000-DPPE DOPC-Chol liposomes + MGC, 5 mg/mL | 109/0.19 | +5.4 |
| CRX-601/5 mol % MPEG-5000-DPPE DOPC-Chol liposomes + MGC, 10 mg/mL | 122/0.22 | +3.4 |
| CRX-601/15 mol % MPEG-5000-DPPE DOPC-Chol liposomes | 96/0.18 | −1.2 |
| CRX-601/15 mol % MPEG-5000-DPPE DOPC-Chol liposomes + MGC, 0.1 mg/mL | 99/0.20 | −6.3 |
| CRX-601/15 mol % MPEG-5000-DPPE DOPC-Chol liposomes + MGC, 0.2 mg/mL | 93/0.14 | −0.8 |
| CRX-601/15 mol % MPEG-5000-DPPE DOPC-Chol liposomes + MGC, 0.3 mg/mL | 96/0.19 | 2.1 |
| CRX-601/15 mol % MPEG-5000-DPPE DOPC-Chol liposomes + MGC, 0.4 mg/mL | 100/0.19 | −4.0 |
| CRX-601/15 mol % MPEG-5000-DPPE DOPC-Chol liposomes + MGC, 0.5 mg/mL | 95/0.19 | 0.0 |
| CRX-601/15 mol % MPEG-5000-DPPE DOPC-Chol liposomes + MGC, 0.6 mg/mL | 101/0.18 | −2.9 |
| CRX-601/15 mol % MPEG-5000-DPPE DOPC-Chol liposomes + MGC, 0.7 mg/mL | 101/0.19 | −0.4 |
| CRX-601/15 mol % MPEG-5000-DPPE DOPC-Chol liposomes + MGC, 0.8 mg/mL | 100/0.18 | −0.1 |
| CRX-601/15 mol % MPEG-5000-DPPE DOPC-Chol liposomes + MGC, 0.9 mg/mL | 108/0.17 | −2.0 |
| CRX-601/15 mol % MPEG-5000-DPPE DOPC-Chol liposomes + MGC, 1 mg/mL | 109/0.14 | −0.9 |
| CRX-601/15 mol % MPEG-5000-DPPE DOPC-Chol liposomes + MGC, 2 mg/mL | 108/0.20 | +0.1 |
| CRX-601/15 mol % MPEG-5000-DPPE DOPC-Chol liposomes + MGC, 3 mg/mL | 109/0.18 | +0.3 |
| CRX-601/15 mol % MPEG-5000-DPPE DOPC-Chol liposomes + MGC, 4 mg/mL | 107/0.21 | +0.5 |
| CRX-601/15 mol % MPEG-5000-DPPE DOPC-Chol liposomes + MGC, 5 mg/mL | 111/0.21 | −1.0 |
| CRX-601/15 mol % MPEG-5000-DPPE DOPC-Chol liposomes + MGC, 10 mg/mL | 114/0.22 | +3.3 |
| CRX-601/15 mol % MPEG-2000-DSPE DOPC-Chol liposomes | 99/0.17 | |
| CRX-601/15 mol % MPEG-2000-DSPE DOPC-Chol liposomes + MGC, 0.1 mg/mL | 102/0.19 | −12.3 |
| CRX-601/15 mol % MPEG-2000-DSPE DOPC-Chol liposomes + MGC, 0.2 mg/mL | 103/0.17 | −8.1 |
| CRX-601/15 mol % MPEG-2000-DSPE DOPC-Chol liposomes + MGC, 0.3 mg/mL | 105/0.19 | −4.6 |
| CRX-601/15 mol % MPEG-2000-DSPE DOPC-Chol liposomes + MGC, 0.4 mg/mL | 107/0.20 | −4.7 |
| CRX-601/15 mol % MPEG-2000-DSPE DOPC-Chol liposomes + MGC, 0.6 mg/mL | 111/0.15 | 0.2 |
| CRX-601/15 mol % MPEG-2000-DSPE DOPC-Chol liposomes + MGC, 0.7 mg/mL | 115/0.19 | −2.2 |
| CRX-601/15 mol % MPEG-2000-DSPE DOPC-Chol liposomes + MGC, 0.8 mg/mL | 109/0.18 | −2.5 |
| CRX-601/15 mol % MPEG-2000-DSPE DOPC-Chol liposomes + MGC, 0.9 mg/mL | 114/0.18 | −2.3 |
| CRX-601/15 mol % MPEG-2000-DSPE DOPC-Chol liposomes + MGC, 0.5 mg/mL | 107/0.17 | −5.2 |
| CRX-601/15 mol % MPEG-2000-DSPE DOPC-Chol liposomes + MGC, 1 mg/mL | 116/0.20 | −1.7 |
| CRX-601/15 mol % MPEG-2000-DSPE DOPC-Chol liposomes + MGC, 2 mg/mL | 110/0.18 | +1.8 |
| CRX-601/15 mol % MPEG-2000-DSPE DOPC-Chol liposomes + MGC, 3 mg/mL | 115/0.20 | +6.5 |
| CRX-601/15 mol % MPEG-2000-DSPE DOPC-Chol liposomes + MGC, 4 mg/mL | 124/0.26 | +11.0 |
| CRX-601/15 mol % MPEG-2000-DSPE DOPC-Chol liposomes + MGC, 5 mg/mL | 120/0.23 | +4.5 |
| CRX-601/15 mol % MPEG-2000-DSPE DOPC-Chol liposomes + MGC, 10 mg/mL | 139/0.26 | +9.6 |
| CRX-601/30 mol % MPEG-5000-DPPE DOPC-Chol liposomes | 87/0.20 | −7.4 |
| CRX-601/30 mol % MPEG-5000-DPPE DOPC-Chol liposomes + MGC, 0.2 mg/mL | 85/0.21 | −2.0 |
| CRX-601/30 mol % MPEG-5000-DPPE DOPC-Chol liposomes + MGC, 0.4 mg/mL | 85/0.21 | −1.7 |
| CRX-601/30 mol % MPEG-5000-DPPE DOPC-Chol liposomes + MGC, 1 mg/mL | 83/0.20 | 0.4 |
| CRX-601/30 mol % MPEG-5000-DPPE DOPC-Chol liposomes + MGC, 2 mg/mL | 94/0.21 | −1.4 |
| CRX-601/30 mol % MPEG-5000-DPPE DOPC-Chol liposomes + MGC, 3 mg/mL | 97/0.22 | +0.1 |
| CRX-601/30 mol % MPEG-5000-DPPE DOPC-Chol liposomes + MGC, 4 mg/mL | 86/0.22 | +1.3 |
| CRX-601/30 mol % MPEG-5000-DPPE DOPC-Chol liposomes + MGC, 5 mg/mL | 87/0.24 | +1.4 |
| CRX-601/30 mol % MPEG-5000-DPPE DOPC-Chol liposomes + MGC, 0.6 mg/mL | 78/0.23 | −2.5 |
| CRX-601/30 mol % MPEG-5000-DPPE DOPC-Chol liposomes + MGC, 0.8 mg/mL | 76/0.21 | −1.4 |
| CRX-601/30 mol % MPEG-5000-DPPE DOPC-Chol liposomes + MGC, 10 mg/mL | 99/0.27 | −1.8 |
| CRX-601/30 mol % MPEG-2000-DSPE DOPC-Chol liposomes | 86/0.16 | −3.8 |
| CRX-601/30 mol % MPEG-2000-DSPE DOPC-Chol liposomes + MGC, 0.2 mg/mL | 82/0.18 | −3.8 |
| CRX-601/30 mol % MPEG-2000-DSPE DOPC-Chol liposomes + MGC, 0.4 mg/mL | 89/0.18 | −2.2 |
| CRX-601/30 mol % MPEG-2000-DSPE DOPC-Chol liposomes + MGC, 0.6 mg/mL | 88/0.16 | −4.8 |
| CRX-601/30 mol % MPEG-2000-DSPE DOPC-Chol liposomes + MGC, 0.8 mg/mL | 82/0.20 | −4.4 |
| CRX-601/30 mol % MPEG-2000-DSPE DOPC-Chol liposomes + MGC, 1 mg/mL | 92/0.22 | −0.7 |
| CRX-601/30 mol % MPEG-2000-DSPE DOPC-Chol liposomes + MGC, 2 mg/mL | 110/0.19 | −0.1 |
| CRX-601/30 mol % MPEG-2000-DSPE DOPC-Chol liposomes + MGC, 3 mg/mL | 110/0.23 | +2.0 |
| CRX-601/30 mol % MPEG-2000-DSPE DOPC-Chol liposomes + MGC, 4 mg/mL | 107/0.22 | +2.8 |
| CRX-601/30 mol % MPEG-2000-DSPE DOPC-Chol liposomes + MGC, 5 mg/mL | 112/0.22 | +7.0 |
| CRX-601/30 mol % MPEG-2000-DSPE DOPC-Chol liposomes + MGC, 10 mg/mL | 122/0.25 | +10.3 |

Chitosan Oligosaccharide Lactate

Investigations were made in the same way as in with methylglycol chitosan (MGC) above except that Chitosan oligosaccharide lactate was used in place of methylglycol chitosan. All tested compositions with liposomes from Example 1 had aggregation. All other formulations remained stable in suspension.

Glycol Chitosan

Investigations were made in the same way as with methylglycol chitosan (MGC) above except that glycol chitosan was used in place of methylglycol chitosan. All tested compositions with liposomes from Example 1 and 2 had aggregation. Liposomes from Example 3 and 4 remained stable in suspension.

Chitosan Coated Liposomes:

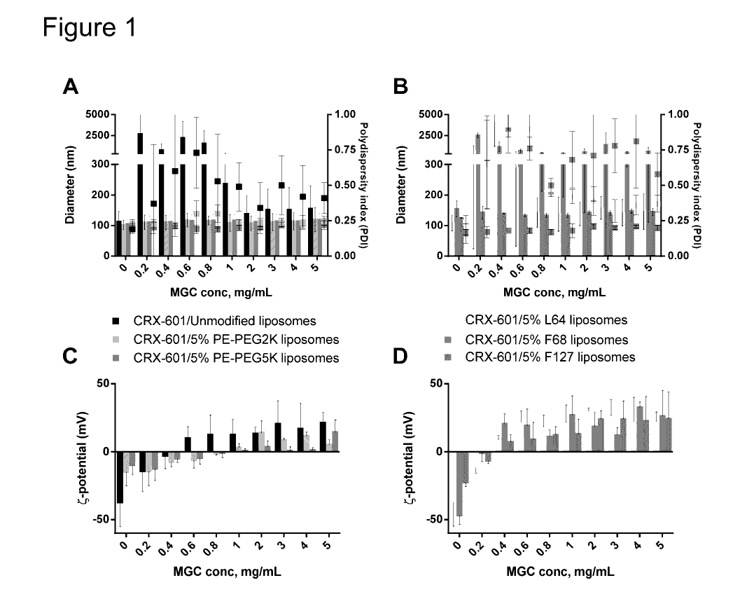

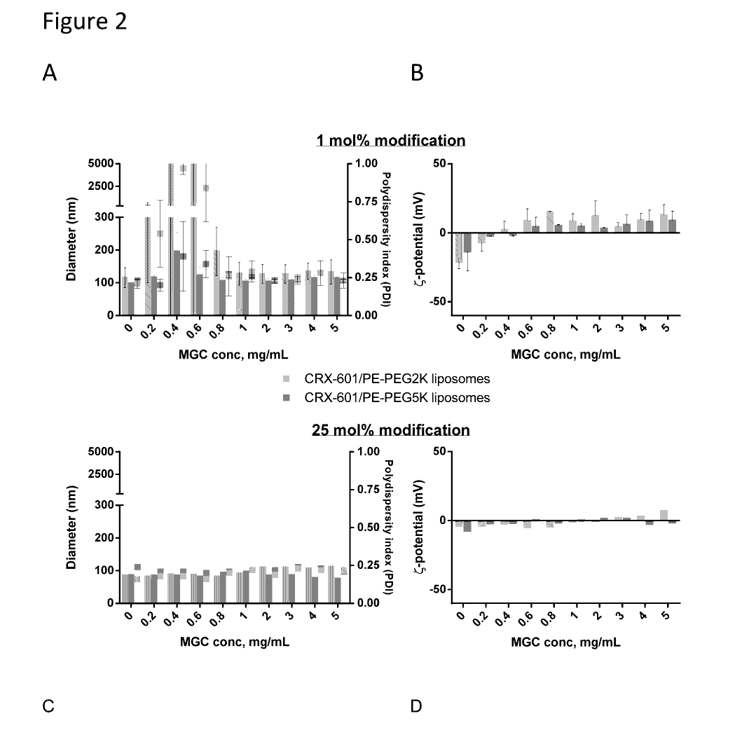

Chitosan coated liposome formulations were prepared by admixing unmodified, phospholipid-PEG modified, or Pluronic modified CRX-601 liposomes with the chitosan derivative and evaluated for changes in size and ζ-potential. When combined with MGC, unmodified liposomes exhibited aggregation, leading to precipitation, at 0.4-2 mg/mL MGC, indicated in FIG. 1A as particles exhibiting size in the μm range. At MGC concentrations exceeding 2 mg/mL, the formulations appeared colloidally stable initially but tended to aggregate over 1-4 days. PE-PEG2K and PE-PEG5K modified liposomes with 5 mol % modification were colloidally stable in presence of MGC with no major change in size at any of concentrations tested (FIG. 1A). Reversal in ζ-potential from a negative potential to positive, occurred at approximately 0.5 mg/ml MGC with unmodified liposomes and 1 mg/mL with 5% PE-PEG2K or PE-PEG5K modification. Modification with as little as 1 mol % PE-PEG2K or PE-PEG5K was demonstrated to provide sufficient protection against MGC induced aggregation at most concentrations (FIG. 2A). At 1% modification, PE-PEG5K liposomes were more resistant to MGC induced aggregation than PE-PEG2K modified liposomes. At 0.4-0.6 mg/mL MGC, no major change in particle size or PDI were observed with 1% PE-PEG5K modification but an increase in size to the μm range were observed with 1% PE-PEG2K modification. Modification of up to 25% did not result in any destabilization/aggregation in presence of MGC (FIG. 2B).

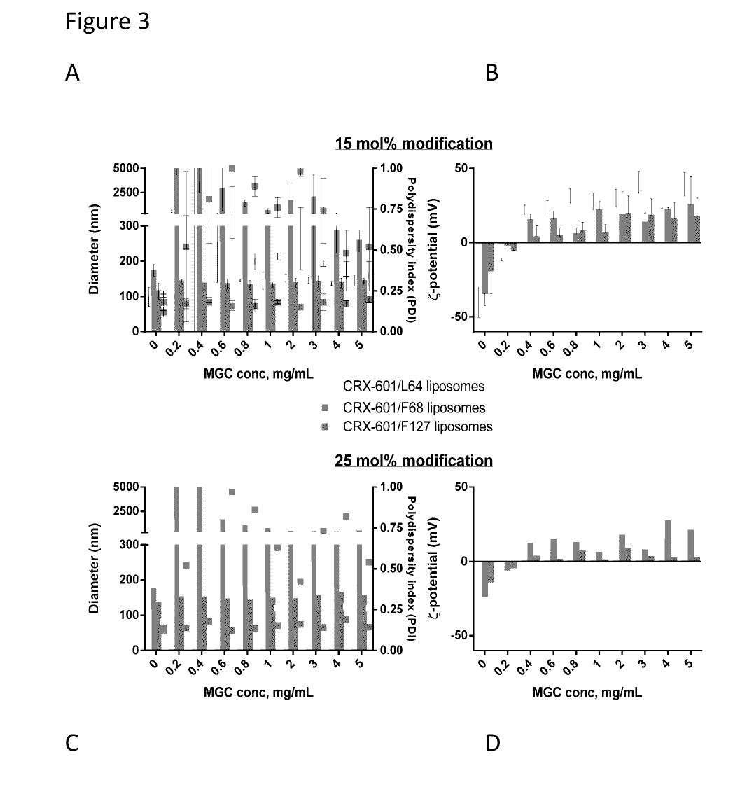

Amongst Pluronic modified liposomes, F127 modified liposomes were the most stable, exhibiting no visible aggregation or increase in polydispersity over the complete range of MGC concentrations evaluated (FIG. 1B). Increase in particle size was about 10-30 nm, and reversal of ζ-potential from a net negative to positive potential occurred at MGC concentrations≧0.4 mg/mL. F127 modified liposomes at 15 and 25% modification were similarly stable in presence of MGC (FIG. 3A), but at 1% modification indicated aggregation (data not shown). Liposomes with 5 mol % L64 modification when combined with MGC, showed an increase in size and polydispersity at 0.2-0.8 mg/mL MGC (FIG. 1B), corresponding to complete neutralization of liposome surface charge. At 1 mg/mL MGC, similar to unmodified liposomes, the L64 modified liposomes appeared stable initially but tended to aggregate and precipitate over time (FIG. 1B). F68 modified liposomes were the least stable, and caused instantaneous precipitation with MGC at all tested concentrations. Similar trends were observed with liposomes with higher Pluronic modification of 15 and 25% (FIG. 3). Hence, the order of stability of Pluronic modified liposomes in presence of MGC was F127>L64>F68.

The summary of stability evaluation for these liposomal formulations in the presence of chitosan derivatives, MGC, GC and CO, is shown in Table 7. Overall, amongst all tested chitosan derivatives, least aggregation was observed with MGC. Phospholipid-PEG modified liposomes were more stable against chitosan induced aggregation than Pluronic liposomes. Only the formulations which exhibited a significant reduction in charge (phospholipid-PEG or Pluronic F127 modified liposomes) were resistant to chitosan induced aggregation. PE-PEG5K liposomes were more stable than PE-PEG2K liposomes, as evident by lack of any change in size/PDI in presence of MGC at 1% modification and improved stability in the presence of GC and CO.

TABLE 7
Stability summary of unmodified, phosholipid-PEG, and
Pluronic liposomes with varying degree of modification
against chitosan derivative induced aggregation^{a}

| Formulation | +MGC^{b} | +GC^{c} | +CO^{d} |
|---|---|---|---|
| CRX-601 unmodified | Aggregation | Aggregation | Aggregation |
| liposomes | | | |

Phospholipid-PEG modified liposomes

| | | | |
|---|---|---|---|
| CRX-601/PE-PEG2K | Stable at ≧1% | Aggregation | Stable at ≧5% |
| liposome | modification^{e} | | modification |
| CRX-601/PE-PEG5K | Stable at ≧1% | Stable at ≧5% | Stable at ≧1% |
| liposome | modification | modification | modification |

Pluronic modified liposomes

| | | | |
|---|---|---|---|
| CRX-601/L64 | Aggregation | Aggregation | Aggregation |
| liposomes | | | |
| CRX-601/F68 | Aggregation | Aggregation | Aggregation |
| liposomes | | | |
| CRX-601/F127 | Stable at ≧5% | Aggregation | Stable at ≧5% |
| liposome | modification | | modification |

^{a}The lowest modification tested was 1 mol %
^{e}Partial aggregation 0.2-0.6 mg/mL
^{b}MGC: Methylglycol chitosan,
^{c}GC: Glycol chitosan,
^{d}CO: Chitosan oligosaccharide lactate

### Example 10

### Rabbit Pyrogen Test

The pyrogen test is used here as a surrogate measure of CRX-601 incorporation into modified liposomes from Example 1-6 and as a measure of their stability in biological milieu. The test was performed at Pacific Biolabs (Hercules, Calif.) as per their SOP 16E-02, which follows procedures outlined in USP<151>. All formulations from Example 1-6 lacked pyrogenicity up to a concentration of at least 250 ng CRX-601/kg animal body weight, except for formulations from Example 7 (poloxamer 188 modified liposomes) and Example 8 (poloxamer 184 modified liposomes). This lack of pyrogenicity up to 250 ng/kg corresponding to a 100 fold improvement over free CRX-601 (max non-pyrogenic dose of 2.5 ng/kg), and indicates a >99% incorporation of CRX-601 into the liposome bilayer. The individual temperature increases from three rabbits per test are indicated in table 8.

TABLE 8
Representative rabbit pyrogen test measurements for formulations described in Example
2, 4, 6, and 9. Values in parenthesis are maximum temperature change for three animals
during the testing period. A temperature rise of 0.5° C. or more is considered a
pyrogenic response. The symbols P and F indicate a ‘Pass’ or ‘Fail’ response respectively.

| | Max Temp. Rise | Max Temp. Rise |
|---|---|---|
| | observed at any time | observed at any time |
| | interval (compared to | interval (compared to |
| | controls) at a dose of | controls) at a dose of |
| Formulation | 250 ng/kg | 500 ng/kg |
| CRX-601 5% MPEG-2000-DSPE DOPC Chol liposome | P (0.1° C., 0.4° C., 0.0° C.) | F (0.5° C., 0.4° C., 0.5° C.) |
| CRX-601 15% MPEG-2000-DSPE DOPC Chol | P (0.0° C., 0.1° C., 0.0° C.) | P (0.0° C., 0.0° C., 0.2° C.) |
| liposome | | |
| CRX-601 30% MPEG-2000-DSPE DOPC Chol | P (0.1° C., 0.0° C., 0.0° C.) | P (0.1° C., 0.3° C., 0.0° C.) |
| liposome | | |
| CRX-601 5% MPEG-5000-DPPE DOPC Chol liposome | P (0.0° C., 0.0° C., 0.0° C.) | P (0.2° C., 0.1° C., 0.0° C.) |
| CRX-601 15% MPEG-5000-DPPE DOPC Chol | P (0.3° C., 0.2° C., 0.0° C.) | P (0.0° C., 0.0° C., 0.4° C.) |
| liposome | | |
| CRX-601 30% MPEG-5000-DPPE DOPC Chol | P (0.3° C., 0.0° C., 0.3° C.) | P (0.3° C., 0.0° C., 0.0° C.) |
| liposome | | |
| CRX-601 5% MPEG-2000-DSPE DOPC Chol liposome, | | P (0.0° C., 0.2° C., 0.1° C.) |
| MGC 5 mg/mL | | |
| CRX-601 5% MPEG-5000-DPPE DOPC Chol liposome, | | P (0.0° C., 0.2° C., 0.1° C.) |
| MGC 5 mg/mL | | |
| CRX-601 5% MPEG-2000-DSPE DOPC Chol liposome, | | P (0.0° C., 0.0° C., 0.2° C.) |
| CL 5 mg/mL | | |
| CRX-601 5% MPEG-5000-DPPE DOPC Chol liposome, | | P (0.4° C., 0.0° C., 0.4° C.) |
| CL 5 mg/mL | | |
| CRX-601 5% poloxamer 407 DOPC liposome | P (0.3° C., 0.3° C., 0.1° C.) | F (0.3° C., 0.3° C., 1.0° C.) |
| CRX-601 10% poloxamer 407 DOPC liposome | P (0.2° C., 0.0° C., 0.0° C.) | F (0.0° C., 1.1° C., 0.0° C.) |
| CRX-601 15% poloxamer 407 DOPC liposome | P (0.2° C., 0.0° C., 0.1° C.) | P (0.0° C., 0.3° C., 0.0° C.) |
| CRX-601/15% poloxamer 188 DOPC liposome | | F (0.5° C., 0.4° C., 0.8° C.) |
| CRX-601/25% poloxamer 188 DOPC liposome | | F (0.3° C., 0.7° C., 0.4° C.) |
| CRX-601/15% poloxamer 184 DOPC liposome | F (0.3° C., 0.6° C., 0.7° C.) | F (0.8° C., 0.8° C., 0.4° C.) |
| CRX-601/25% poloxamer 184 DOPC liposome | F (0.9° C., 0.5° C., 0.9° C.) | F (0.5° C., 0.6° C., 0.9° C.) |
| Methylglycol chitosan (MGC) | | P (0.0° C., 0.0° C., 0.1° C.) |
| Chitosan lactate (CL) | | P (0.0° C., 0.0° C., 0.0° C.) |

Abbreviations:
Methylglycol chitosan (MGC);
Chitosan oligosaccharide lactate (CL)

### Example 11

### Mouse Sublingual Vaccination and Determination of Specific Antibody Responses

Female BALB/c mice (6 to 8 weeks of age) obtained from Charles River Laboratories (Wilmington, Mass.) were used for these studies. Mice anesthetized by intraperitoneal (i.p) administration of ketamine (100 mg/kg) and xylazine (10 mg/kg) were given vaccine by sublingual administration (5-6 μLs). All mice were vaccinated on days 0, 21 and 42 with the 5 μg CRX-601 in the liposomes formulation admixed with 1 or 1.5 μg HA/mouse using the influenza antigen A/Victoria/210/2009 H3N2. Serum was harvested on day 36 (14dp2) under anesthesia, on day 56 (14dp3) mice were sacrificed and a final harvest of vaginal washes, tracheal washes and serum were collected. All animals were used in accordance with guidelines established by the U.S. Department of Health and Human Services Office of Laboratory Animal Welfare and the Institutional Animal Care and Use Committee at GSK Biologicals, Hamilton, Mont.

Specific antibody responses were measured by two independent immunoassays, the enzyme linked immunosorbent assay (ELISA) and the influenza hemagglutinin inhibition (HI) assay.

ELISA was performed using split flu coated 96 well plates (Nunc Maxisorp) and detecting the bound immunoglobins from the added serum or tracheal wash or vaginal wash samples using peroxidase linked goat anti-mouse IgG, IgG1, IgG2a or IgA. This was followed by addition of an enzyme specific chromogen, which resulted in color intensity directly proportional to the amount of specific antiflu IgGs/IgAs contained in the serum. The optical density was read at 450 nm.

HI assay was performed by evaluating inhibition of chicken or rooster RBCs upon exposure to flu virus in presence of mouse serum. The reciprocal of the last dilution of influenza virus which resulted in complete or partial agglutination of RBCs was used to calculate the HI titer and expressed as HA units/50 μl of sera.

### Example 12

### Mouse Sublingual Vaccination with Liposomes Modified with MPEG-2000-DSPE or MPEG-5000-DPPE (NIH #162)

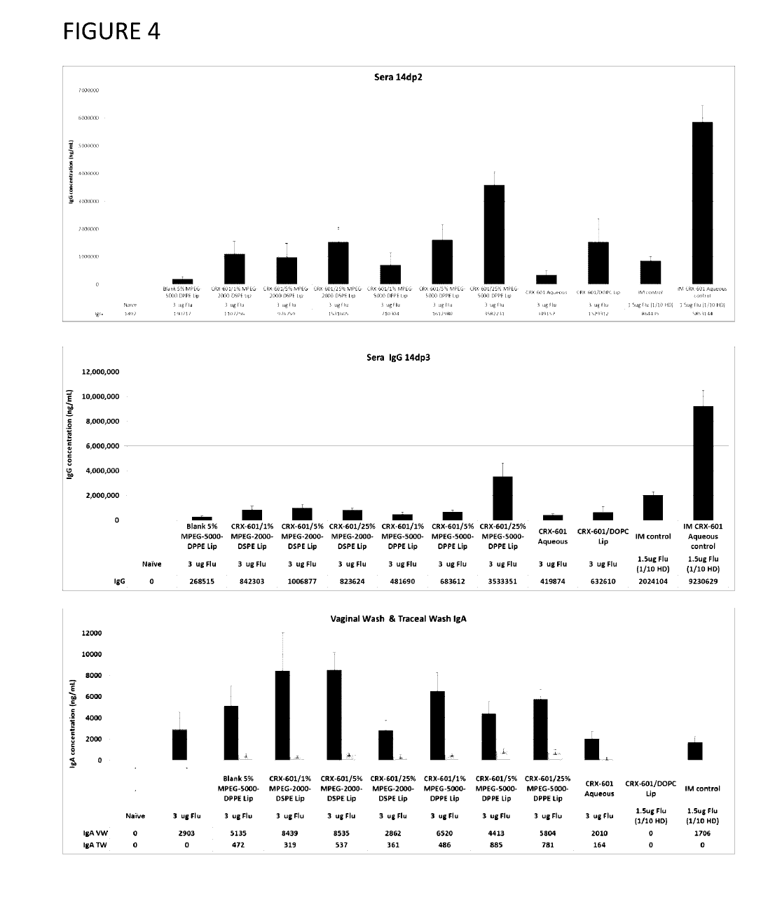

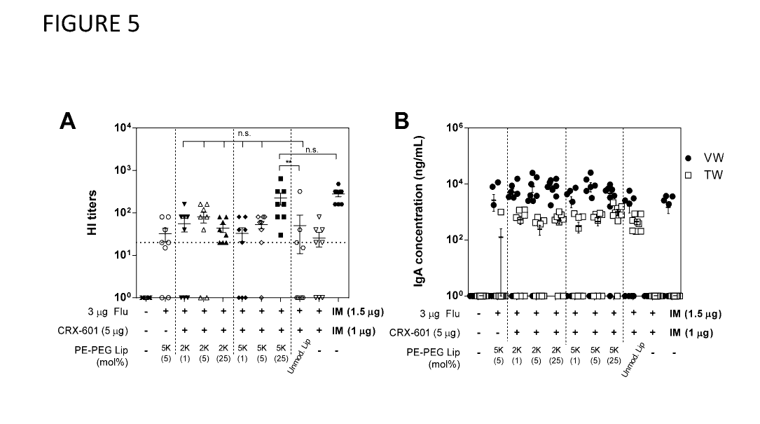

The mice were vaccinated using the procedure outlined in Example 11 with 1, 5, and 25 mole % MPEG-2000-DSPE or MPEG-5000-DPPE modified liposomes from Example 1-4. The serum IgG titers 14 days post-secondary and post-tertiary vaccinations are shown in FIG. 4 (and also with HI titers in FIG. 5.) Amongst the sublingual treatment groups, titers were highest in mice receiving CRX-601 in 25% MPEG-5000-DPPE liposome treatment group, significantly higher than CRX-601 aqueous or CRX-601 unmodified liposome treatment groups.

### Example 13

### Mouse Sublingual Vaccination with Liposomes Modified with Poloxamers 407 (NIH #158)

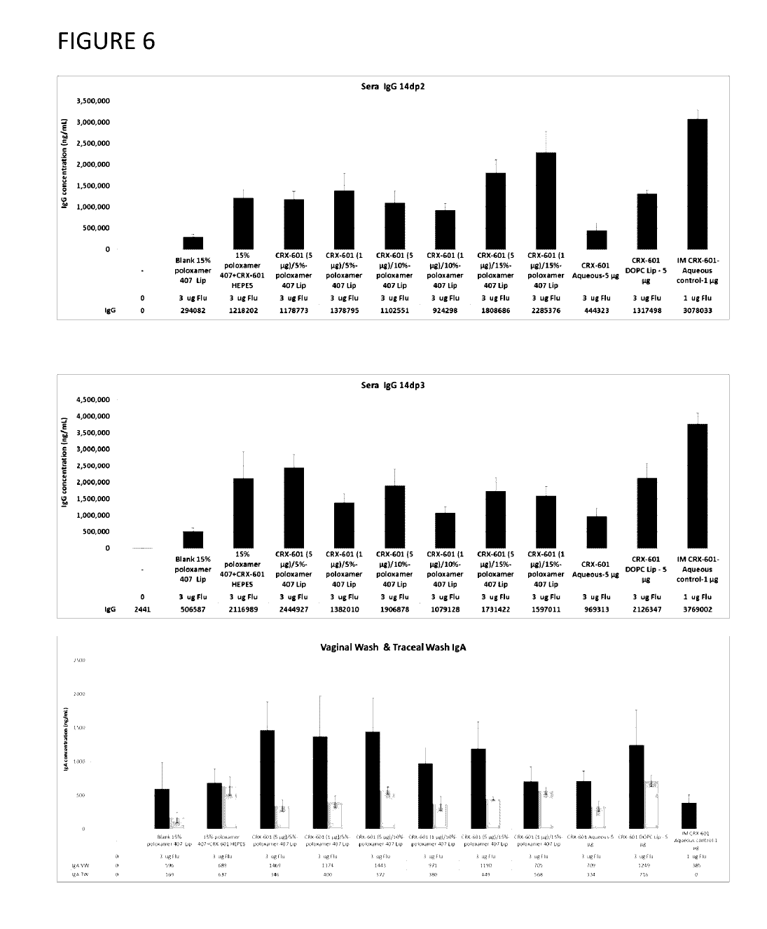

The mice were vaccinated using the procedure outlined in Example 11 with 5, 10 and 15 mole % poloxamer 407 modified liposomes from Example 5-6. The serum IgG titers 14 days post-secondary and post-tertiary vaccinations are shown in FIG. 6. Amongst the sublingual treatment groups, titers were highest in mice receiving CRX-601 in 15% poloxamer 407 modified liposome treatment group (post-seondary), significantly higher than CRX-601 aqueous or CRX-601 unmodified liposome treatment groups.

### Example 14

### Mouse Sublingual Vaccination with Liposomes Modified with Poloxamers 407, 188, and 184 (NIH #167)

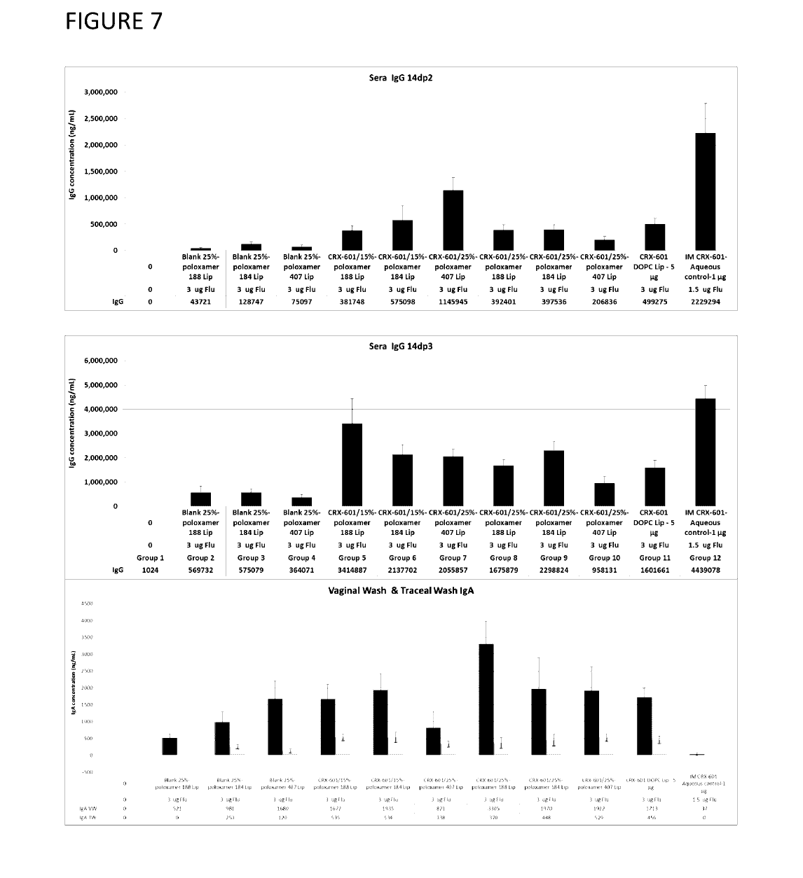

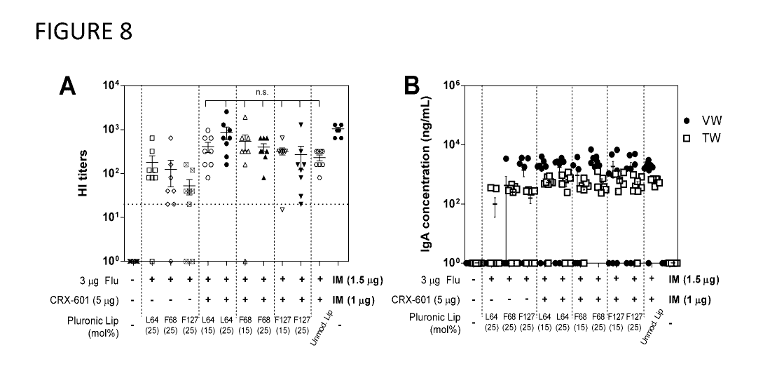

The mice were vaccinated using the procedure outlined in Example 11 with 15 and 25 mole % poloxamer 407, or 188, or 184 modified liposomes from Example 5-8. The serum IgG titers 14 days post-secondary and post-tertiary vaccinations are shown in FIG. 7 (and with HI titres in FIG. 8). Amongst the sublingual treatment groups, titers were highest in mice receiving CRX-601 in 15% poloxamer 188 liposome treatment group. Titers in CRX-601/poloxamer modified liposome treatment groups were in general better than CRX-601 aqueous or CRX-601 unmodified liposome treatment groups.

### Example 15

### Mouse Sublingual Vaccination with Liposomes Modified with MPEG-2000-DSPE or MPEG-5000-DPPE and Methylglycol Chitosan or Chitosan Oligosaccharide Lactate (NIH #163)

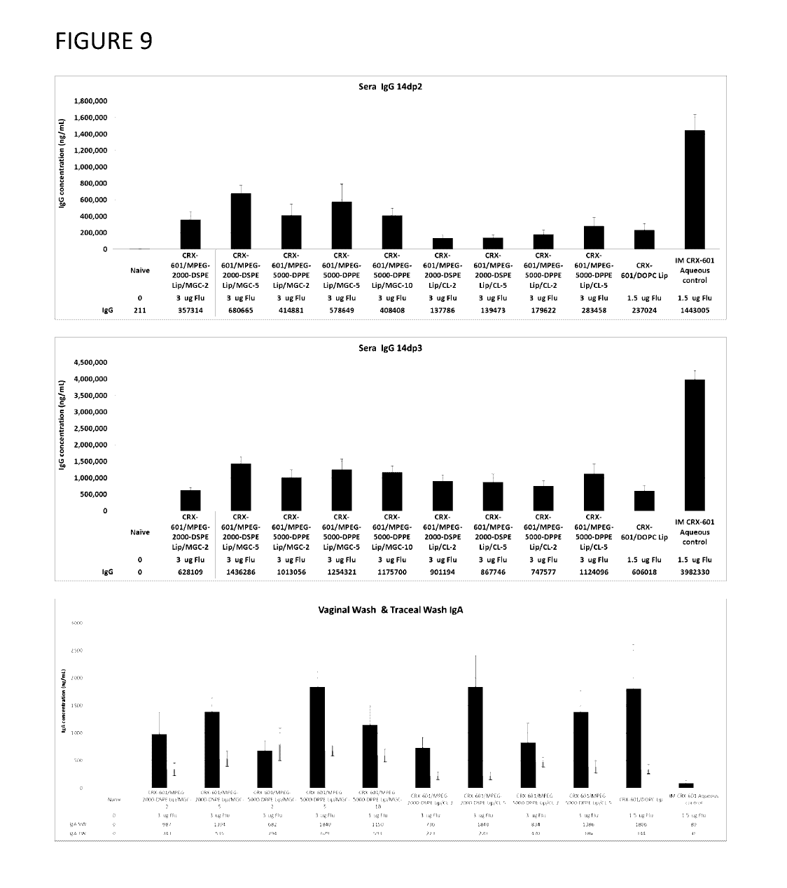

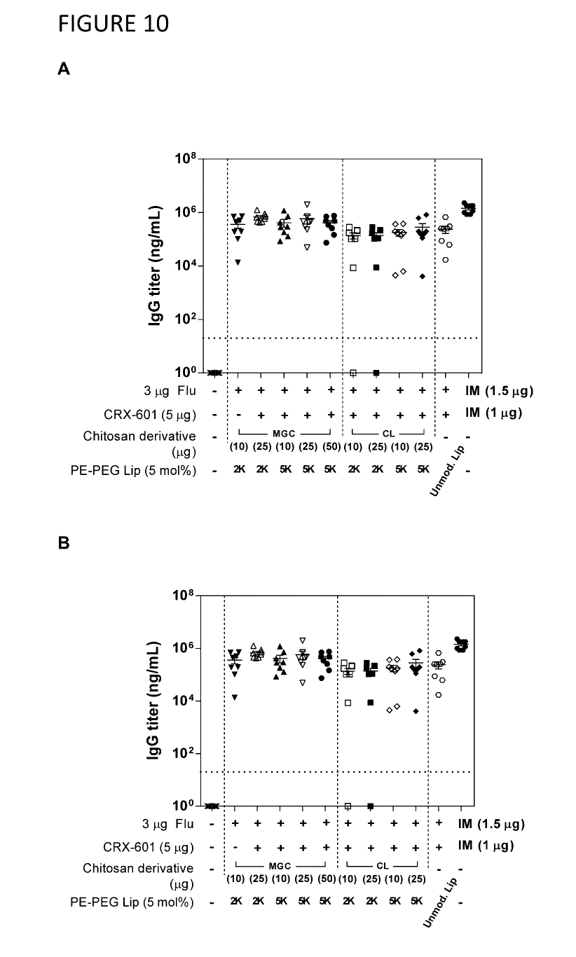

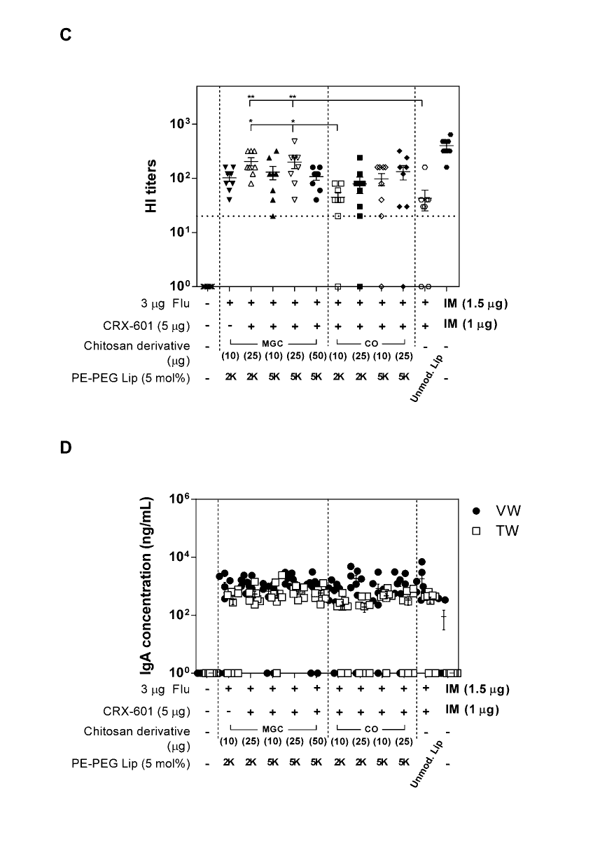

The mice were vaccinated using the procedure outlined in Example 11 with 5 mole % MPEG-2000-DSPE or MPEG-5000-DPPE modified liposomes from Example 2 and 4 formulated with methylglycol chitosan or chitosan oligosaccharide lactate as described in Example 9. The serum IgG titers 14 days post-secondary and post-tertiary vaccinations are shown in FIG. 9 (and also with HI titers in FIG. 10). Amongst the sublingual treatment groups, titers in liposome+methylglycol chitosan treatment groups were in general better than CRX-601 unmodified liposome treatment groups.

### Example 16

### Mouse Sublingual Vaccination with Liposomes Modified with Poloxamers and Methylglycol Chitosan (NIH #164)

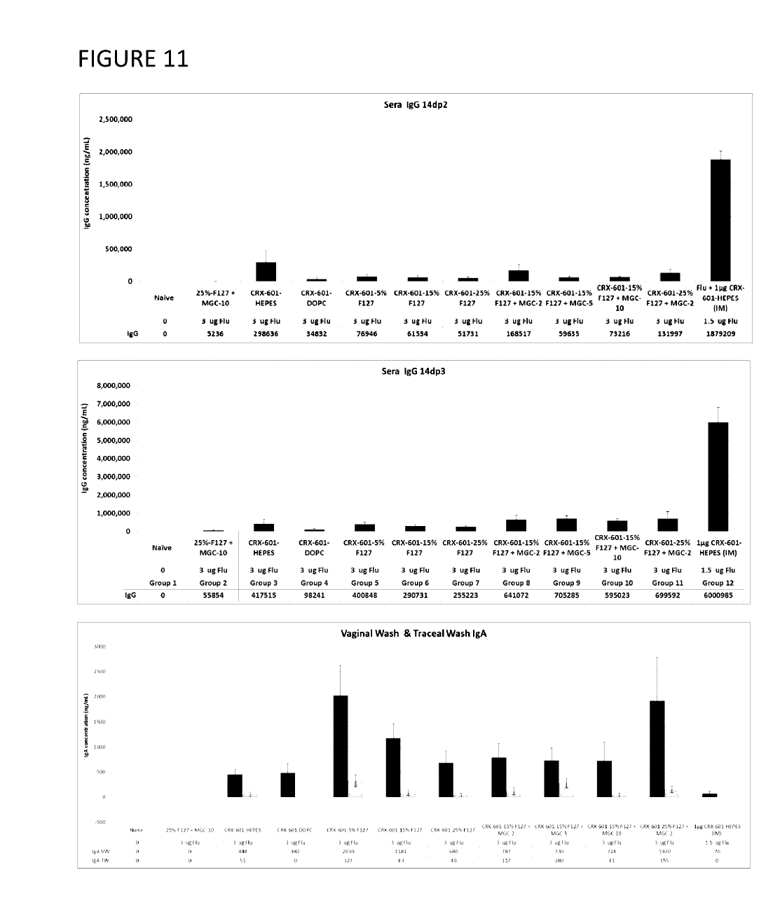

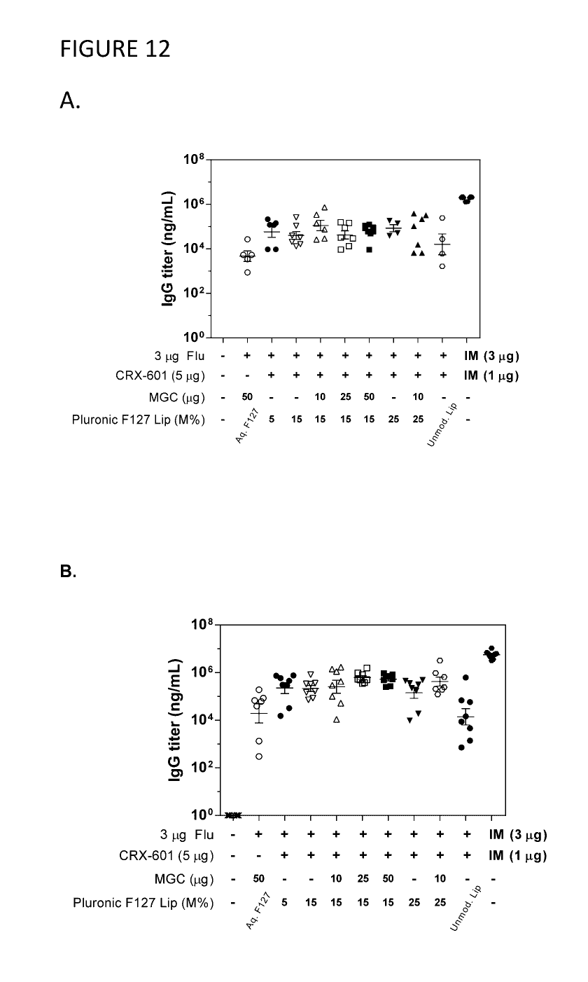

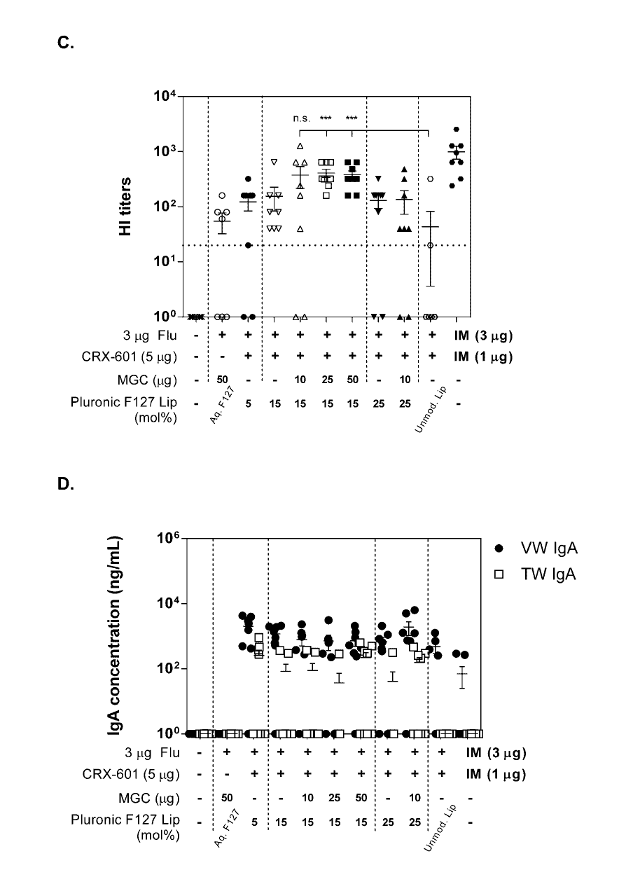

The mice were vaccinated using the procedure outlined in Example 11 with 5, 15 and 25 mole % poloxamer 407 (labeled F127 in FIGS. 11 and 12) modified liposomes from Example 5-8 and 15 or 25 mole % poloxamer 407 liposomes formulated with methylglycol chitosan as described in Example 9. The serum IgG titers 14 days post-secondary and post-tertiary vaccinations are shown in FIG. 11 (and also with HI titers in FIG. 12).

1. A liposomal composition comprising lipids which form a liposomal lipid bilayer, phospholipid-PEG conjugates incorporated into the liposomal lipid bi-layer, and a chitosan or chitosan derivative

2. The liposomal composition of claim 1 further comprising an aminoalkyl glucosaminide phosphate (AGP) and aminoalkanesulfonic buffer.

3. The liposomal composition of claim 2 wherein the lipids of the liposome are DOPC.

4. The liposomal composition of claim 2 wherein the liposomal composition further comprises cholesterol.

5. A liposomal composition comprising lipids which form a liposomal lipid bilayer, PEG copolymers/surfactants such as poloxamers incorporated into the liposomal lipid bi-layer, AGP and an aminoalkanesulfonic buffer.

6. The liposomal composition of claim 1 wherein the lipids of the liposome are DOPC in the absence of cholesterol.

7. The liposomal composition of claim 1 wherein the phospholipid-PEG conjugate is selected from the group consisting of poloxamer 407 (Pluronic® F127); poloxamer 184 (Pluronic® L64); poloxamer 188 (Pluronic® L68).

8. The liposomal composition of claim 1 wherein the phospholipid-PEG conjugate is MPEG-2000-DSPE N-(Carbonyl-methoxypolyethylenglycol-2000)-1,2-distearoyl-sn-glycero-3-phosphoethanolamine sodium salt, or MPEG-5000-DPPE N-(Carbonyl-methoxypolyethylenglycol-5000)-1,2-dipalmitoyl-sn-glycero-3-phosphoethanolamine sodium salt.

9. The liposomal composition of claim 1 wherein the liposomal composition further comprises chitosan.

10. The liposomal composition of claim 2 wherein the amninoalkanesulfonic buffer is selected from the group consisting of HEPES, HEPPS/EPPS, MOPS, MOBS and PIPES.

11. The liposomal composition of claim 2 wherein the AGP is selected from the group consisting of: CRX-601, CRX 602, CRX 527, CRX 547, CRX 526, CRX 529 or CRX 524.

12. The liposomal composition of claim 9 comprising a chitosan, wherein the chitosan is selected from the group consisting of: chitosan oligosaccharide lactate, trimethyl chitosan glycol chitosan, and methylglycol chitosan.

13. A process for improved production of a liposomal composition for mucosal delivery comprising the steps of:
a. dissolving a lipid, such as dioleoyl phosphatidylcholine, phospholipid-PEG
b. conjugate, and AGP in organic solvent,
c. removing the solvent to yield a phospholipid film,
d. adding the film to HEPES buffer or HEPES buffer in saline,
e. dispersing the film into the solution, and
f. extruding the solution successively through polycarbonate filters to form unilamellar liposomes.

14. The process of claim 13 wherein the AGP is CRX-601.

15. The liposomal composition of claim 2 wherein the AGP is CRX-601 is present in an amount less than 10, less than 9, less than 8, less than 7, less than 6, less than 5, less than 4, less than 3, less than 2 or less than 1 mg.

16. The liposomal composition of claim 15 wherein the AGP is CRX-601 is present in an amount between 30 ug/mL and 6 mg/mL.

17. The liposomal composition of claim 1 wherein the liposome is multilamellar

18. The liposomal composition of claim 1 wherein the liposome is 2, 3, 4, 5, 6, 7, 8, 9, or 10 lamellar.

19. The liposomal composition of claim 1 wherein the liposome is unilamellar.

20. The liposomal composition of claim 1 wherein the liposome size will be in the range of 50 nm to 500 nm and in further embodiments 50 nm to 200 nm.

21. The liposomal composition of claim 1 wherein the liposome size will be in the range of about 80-120 nm.

22. The liposomal composition of claim 1 wherein the liposomal structures encloses an aqueous interior.

23. The liposomal composition of claim 1 further comprising a lipid A mimetic, TLR4 ligand, or AGP.

24. The liposomal composition of claim 2 wherein the AGP is compound having the structure set forth in Formula I: 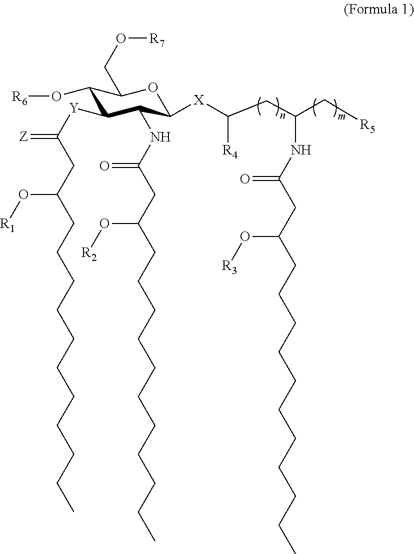
b. wherein
c. m is 0 to 6
d. n is 0 to 4;
e. X is O or S, preferably O;
f. Y is O or NH;
g. Z is O or H;
h. each R₁, R₂, R₃ is selected independently from the group consisting of a C₁₋₂₀ acyl and a C₁₋₂₀ alkyl;
i. R₄ is H or Me;
j. R₅ is selected independently from the group consisting of —H, —OH, —(C₁-C₄) alkoxy, —PO₃R₈R₉, —OPO₃R₈R₉, —SO₃R₈, —OSO₃R₈, —NR₈R₉, —SR₈, —CN, —NO₂, —CHO, —CO₂R₈, and —CONR₈R₉, wherein R₈ and R₉ are each independently selected from H and (C₁-C₄) alkyl; and
k. each R₆ and R₇ is independently H or PO₃H₂.

25.-29. (canceled)
